(19) United States
(12) Patent Application Publication
(10) Pub. No.: US 2018/0140211 A1
(43) Pub. Date: May 24, 2018

(54) WORKOUT INFORMATION DISPLAY METHOD, WORKOUT INFORMATION DISPLAY SYSTEM, SERVER SYSTEM, ELECTRONIC DEVICE, INFORMATION STORAGE MEDIUM, AND PROGRAM

(71) Applicant: SEIKO EPSON CORPORATION, Tokyo (JP)

(72) Inventors: Hironori NAKAZAWA, Matsumoto-shi (JP); Masafumi SATO, Hara-mura (JP); Nobuyuki KITO, Shiojiri-shi (JP); Nonoyama MASAAKI, Kyoto-shi (JP); Kinpara YUKI, Kyoto-shi (JP)

(73) Assignee: SEIKO EPSON CORPORATION, Tokyo (JP)

(21) Appl. No.: 15/795,689

(22) Filed: Oct. 27, 2017

(30) Foreign Application Priority Data
Nov. 22, 2016 (JP) 2016-226414
Jun. 9, 2017 (JP) 2017-113909

(51) Int. Cl. A61B 5/024; A61B 5/11; G06F 17/18; G06T 11/20

(52) U.S. Cl. CPC A61B 5/02438; A61B 5/11; A61B 5/024; G06F 17/18; A61B 5/6824; A61B 2503/10; A61B 2505/09; A61B 2562/0219; G06T 11/206

(57) ABSTRACT

A workout information display method includes: acquiring workout information based on a workout executed by a user; displaying a graph in which a first axis represents a distance or a time and a second axis represents a measurement value of each measurement item of the workout information in a first display region; displaying first to P-th icons for selecting setting of measurement values of first to P-th measurement items on the second axis of the graph in a second display region; and displaying a measurement value of the measurement item corresponding to the icon in regard to at least one of the first to P-th icons.

This application claims the benefit of JP 2017-113909, filed Jun. 9, 2017; and claims the benefit of JP 2016-226414, filed Nov. 22, 2016. The disclosure of the prior applications is hereby incorporated by reference herein in its entirety.

### BACKGROUND

### 1. Technical Field

The present invention relates to a workout information display method, a workout information display system, a server system, an electronic device, an information storage medium, and a program.

### 2. Related Art

In recent years, workouts (in a narrow sense, sports) are generally executed in states in which wearable devices with various sensors such as Global Positioning System (GPS) devices for runners are worn. Data measured with wearable devices are generally browsed and analyzed with smartphone applications, web applications, and other software for browsing and analysis.

For example, JP-A-2015-186593 discloses a system that collects and displays exercise data. In particular, JP-A-2015-186593 discloses a scheme of installing a “Month” button, a “Week” button, or the like, selecting units of periods in which a user desires to browse, and displaying performance information in accordance with the selected units of periods.

JP-A-2011-237289 proposes a system that supports a training according to a purpose of a user. In particular, JP-A-2011-237289 discloses a scheme of searching for a route with the same nature as a specific race course.

In workouts in which wearable devices are worn, various kinds of information such as paces, pitches, strides, and heart rates can be acquired. In general, as an amount of information presented to a user is greater, the size (resolution) of a display image increases. Therefore, it is necessary to perform transition between a plurality of screens or scroll display. However, as in numerical display or graphic display of measured information, there is a lot of information which is preferably browsed simultaneously.

That is, in display of workout information, it is important to efficiently dispose various kinds of information on a restricted display screen. However, in schemes of the related art, disposition of items to be displayed has not been sufficiently disclosed.

### SUMMARY

An advantage of some aspects of the invention is that it provides a workout information display method of performing a process of displaying workout information in a mode appropriate for browsing by a user, a workout information display system, a server system, an electronic device, an information storage medium, and a program (as used herein, the information storage medium does not encompass non-transitory signals).

An aspect of the invention relates to a workout information display method including: acquiring workout information based on a workout executed by a user; displaying a graph in which a first axis represents a distance or a time and a second axis represents a measurement value of each measurement item of the workout information in a first display region; displaying first to P-th icons (where P is an integer equal to or greater than 2) for selecting setting of measurement values of first to P-th measurement items on the second axis of the graph in a second display region when there are the first to P-th measurement items as the measurement items; and displaying a measurement value of the measurement item corresponding to the icon in regard to at least one of the first to P-th icons.

According to the aspect of the invention, the graph is displayed in the first display region, an icon for setting a given axis of the graph is displayed in the second display region, and the measurement value is displayed in the icon.

In this way, since the measurement value (numerical value) can be displayed in the icon for the axis setting, display target information can be efficiently disposed on the screen.

In the aspect of the invention, when a point or a range is designated in the display of the graph, the first to P-th measurement values in the designated point or range may be displayed in regard to the first to P-th icons. When no point or no range is designated in the display of the graph, statistical values obtained through a statistical process for the first to P-th measurement values during a workout period may be displayed in regard to the first to P-th icons.

In this way, information displayed in the icon can be changed in response to an operation on the graph.

In the aspect of the invention, the first to P-th measurement values may include at least one measurement value of a pace, a pitch, a stride, altitude information, a heart rate, and consumed calories in the workout.

In this way, various measurement values can be displayed as in graph and numerical values.

In the aspect of the invention, an operation by the user may be received and the graph and an adjustment object for changing a range of the graph displayed in the first display region may be displayed in the first display region side by side. A length from a starting point to an ending point of the range may be changed by changing at least one of the starting point and the ending point of the range when a first operation by the user on the adjustment object is received. The starting point and the ending point may be changed while keeping the length from the starting point to the ending point of the range when a second operation by the user on the adjustment object is received.

In this way, a range of data (a distance range or a time range) which is a display target of the graph can be flexibly adjusted based on a user operation on the adjustment object.

In the aspect of the invention, map information associated with the workout may be displayed in a third display region.

In this way, the map information can be displayed.

In the aspect of the invention, lap information of the workout may be displayed in a fourth display region.

In this way, the lap information can be displayed.

In the aspect of the invention, the first, second, third, and fourth display regions may be arranged on one display screen.

In this way, the graph, the icon, the map information, and the lap information can be displayed in a mode in which a list property is high.

In the aspect of the invention, the fourth display region may be disposed to overlap the third display region.

In this way, the map information and the lap information can be displayed in the mode in which a list property is high (a mode in which comparison is easy).

In the aspect of the invention, the fourth display region may be switched between non-display and the overlap display on the third display region in response to the received operation by the user.

In this way, visibility of the map information can be improved depending on a situation.

In the aspect of the invention, when an expansion operation by the user is received, the third display region may be expanded to be displayed and the first and second display regions may be moved in a predetermined direction.

In this way, the visibility of the map information can be improved depending on a situation. At this time, a relative positional relation between the graph and the display region of the icon can be appropriately adjusted.

In the aspect of the invention, when the third display region is expanded to be displayed, at least one of the number of pieces of lap information and an amount of information displayed in the fourth display region may be increased further than when the third display region is not expanded to be displayed.

In this way, information presented as the lap information can be increased at the time of expanding and displaying the third display region.

Another aspect of the invention relates to a workout information display method including: acquiring map information associated with a workout executed by a user and lap information of the workout as workout information based on the workout; and disposing and displaying the lap information to overlap the map information.

According to the aspect of the invention, the map information and the lap information of the workout are displayed to overlap with each other. In this way, the map information and the lap information can be displayed in the mode in which a list property is high (a mode in which comparison is easy).

In the aspect of the invention, the lap information may be switched between non-display and the overlap display on the map information in response to the received operation by the user.

In this way, the visibility of the map information can be improved depending on a situation.

Still another aspect of the invention relates to a workout information display system including: a reception unit that receives an operation by a user; a storage unit that stores workout information based on a workout executed by the user; and a display unit that displays the workout information. The display unit displays a graph in which a first axis represents a distance or a time and a second axis represents a measurement value of each measurement item of the workout information in a first display region. The display unit displays first to P-th icons (where P is an integer equal to or greater than 2) for selecting setting of measurement values of first to P-th measurement items on the second axis of the graph in a second display region when there are the first to P-th measurement items as the measurement items. The display unit displays a measurement value of the measurement item corresponding to the icon in regard to at least one of the first to P-th icons.

Still another aspect of the invention relates to a workout information display system including: a reception unit that receives an operation by a user; a storage unit that stores workout information based on a workout executed by the user; and a display unit that displays the workout information. The display unit disposes lap information of the workout to overlap map information associated with the workout.

Still another aspect of the invention relates to a server system including: a reception unit that receives operation information by a user; a storage unit that stores workout information based on a workout executed by the user; and a display processing unit that performs a process of displaying the workout information on a display unit. The display processing unit performs a process of displaying a graph in which a first axis represents a distance or a time and a second axis represents a measurement value of each measurement item of the workout information in a first display region. The display processing unit performs a process of displaying first to P-th icons (where P is an integer equal to or greater than 2) for selecting setting of measurement values of first to P-th measurement items on the second axis of the graph in a second display region when there are the first to P-th measurement items as the measurement items. The display processing unit performs a process of displaying a measurement value of the measurement item corresponding to the icon in regard to at least one of the first to P-th icons.

Still another aspect of the invention relates to a server system including: a reception unit that receives an operation by a user; a storage unit that stores workout information based on a workout executed by the user; and a display processing unit that performs a process of displaying the workout information on a display unit. The display processing unit performs a process of disposing lap information of the workout to overlap map information associated with the workout.

Still another aspect of the invention relates to an electronic device including the above-described workout information display system.

Still another aspect of the invention relates to an information storage medium storing a program causing a computer to function as: a reception unit that receives an operation by a user; a storage unit that stores workout information based on a workout executed by the user; and a display processing unit that performs a process of displaying the workout information on a display unit. The display processing unit displays a graph in which a first axis represents a distance or a time and a second axis represents a measurement value of each measurement item of the workout information in a first display region. The display processing unit displays first to P-th icons (where P is an integer equal to or greater than 2) for selecting setting of measurement values of first to P-th measurement items on the second axis of the graph in a second display region when there are the first to P-th measurement items as the measurement items. The display processing unit displays a measurement value of the measurement item corresponding to the icon in regard to at least one of the first to P-th icons.

Still another aspect of the invention relates to an information storage medium storing a program causing a computer to function as: a reception unit that receives an operation by a user; a storage unit that stores workout information based on a workout executed by the user; and a display processing unit that performs a process of displaying the workout information on a display unit. The display processing unit disposes lap information of the workout to overlap map information associated with the workout.

Still another aspect of the invention relates to a program causing a computer to function as: a reception unit that receives an operation by a user; a storage unit that stores workout information based on a workout executed by the user; and a display processing unit that performs a process of displaying the workout information on a display unit. The display processing unit displays a graph in which a first axis represents a distance or a time and a second axis represents a measurement value of each measurement item of the workout information in a first display region. The display processing unit displays first to P-th icons (where P is an integer equal to or greater than 2) for selecting setting of measurement values of first to P-th measurement items on the second axis of the graph in a second display region when there are the first to P-th measurement items as the measurement items. The display processing unit displays a measurement value of the measurement item corresponding to the icon in regard to at least one of the first to P-th icons.

Still another aspect of the invention relates to a program causing a computer to function as: a reception unit that receives an operation by a user; a storage unit that stores workout information based on a workout executed by the user; and a display processing unit that performs a process of displaying the workout information on a display unit. The display processing unit disposes lap information of the workout to overlap map information associated with the workout.

### BRIEF DESCRIPTION OF THE DRAWINGS

The invention will be described with reference to the accompanying drawings, wherein like numbers reference like elements.

FIG. 1 is a diagram illustrating an example of the configuration of a workout information display system.

FIG. 2 is a diagram illustrating an example of the exterior of a wearable device.

FIG. 3 is a diagram illustrating the example of the exterior of the wearable device.

FIG. 4 is a diagram illustrating an example of a system including a workout information display system.

FIG. 5 is a diagram illustrating screen transition.

FIG. 6 is a diagram illustrating an example of a board screen.

FIG. 7 is a diagram illustrating an example of a card disposition region on the board screen.

FIG. 8 is a diagram illustrating an example of a display screen of history information.

FIG. 9 is a diagram illustrating an example of a display screen of history information.

FIG. 10 is a diagram illustrating an example of a display screen of history information.

FIG. 11 is a diagram illustrating an example of a display screen of detailed information.

FIG. 12 is a diagram illustrating an example of a display screen of detailed information.

FIG. 13 is a diagram illustrating an example of a display screen of detailed information.

FIG. 14 is a diagram illustrating an example of a display screen of detailed information.

FIG. 15 is a diagram illustrating an example of a screen on which a list of similar workout information is displayed.

FIG. 16 is a diagram illustrating an example of a comparison screen of selection workout information and similar workout information.

FIG. 17 is a diagram illustrating a process of retrieving similar workout information.

FIG. 18 is a diagram illustrating the process of retrieving the similar workout information.

FIG. 19 is a diagram illustrating the process of retrieving the similar workout information.

FIG. 20 is a diagram illustrating an example of an operation screen used to set similar conditions.

FIG. 21 is a diagram illustrating an example of a calendar screen.

FIG. 22 is a diagram illustrating an example of a display screen of detailed information.

FIG. 23 is a diagram illustrating an example of the display screen of the detailed information.

FIG. 24 is a diagram illustrating an example of the display screen of the detailed information.

### DESCRIPTION OF EXEMPLARY EMBODIMENTS

Hereinafter, embodiments will be described. The embodiments to be described below inappropriately do not limit content of the invention described in the appended claims. All of the configurations to be described in the embodiments are not necessarily requisites of the invention.

### 1. Example of Configuration of System

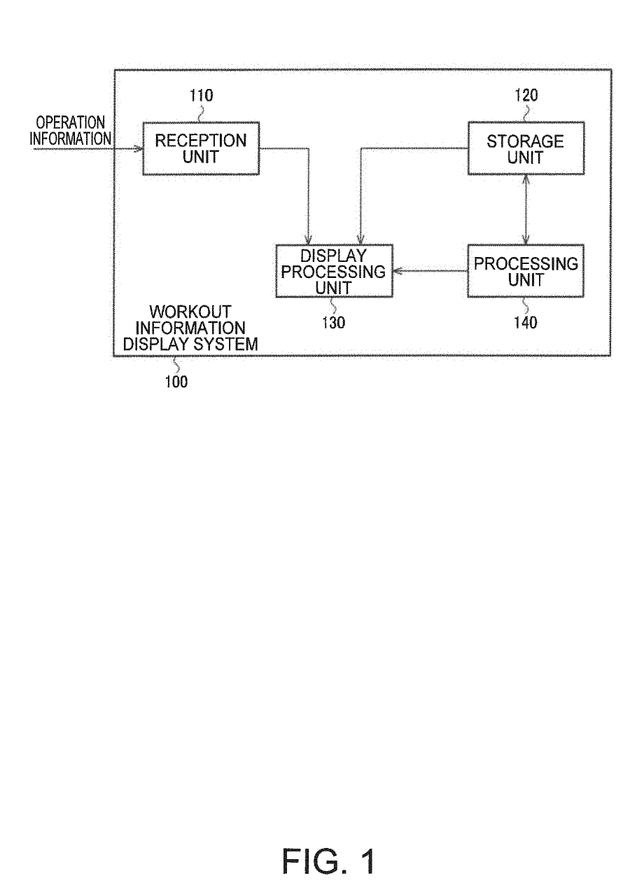

FIG. 1 is a diagram illustrating an example of the configuration of a workout information display system 100 according to an embodiment. The workout information display system 100 includes a reception unit 110 (an operation interface or a communication interface), a storage unit 120 (memory), a display processing unit 130 (processor), a processing unit 140 (processor), and a display unit 150 (display). Here, the workout information display system 100 is not limited to the configuration of FIG. 1. Various modifications can be made by omitting some of constituent elements or adding other constituent elements. For example, the workout information display system 100 may be configured to include a communication unit (communication interface) separately from the reception unit 110. A sensor unit (not illustrated) may be configured to be included. In this case, the sensor unit may be configured to have a function of measuring data regarding a workout and acquire at least some of measurement values related to the workout to be described below. In this case, the measurement values acquired by the sensor unit are added to workout information received by the reception unit 110 to be set as workout information.

The display processing unit 130 and the processing unit 140 may be configured to be integrated so that functions of the display processing unit 130 and the processing unit 140 are realized by one processing unit or an electronic device.

The reception unit 110 receives an operation by a user. Here, the user is a user who browses workout information. The user may be the same as or different from a user who executes a workout. The workout indicates that a body is moving or is training and workout information is various kinds of information acquired by executing workouts.

The user executes operations on various operation units such as a button or a touch panel of a mobile terminal device 300 (smartphone) to be described below and a mouse and a keyboard of an information processing device 400 (personal computer: PC). Then, the reception unit 110 receives operations.

The storage unit 120 is a work area of the processing unit 140 or the like and a function of the storage unit 120 can be realized by a memory such as a random access memory (RAM), a hard disk drive (HDD), or the like. The storage unit 120 stores workout information based on a workout executed by the user.

The display processing unit 130 performs a process of displaying the workout information on the display unit 150. The display unit 150 is a unit that displays various display screens and can be realized by, for example, a liquid crystal display or an organic EL display. Here, the display unit 150 is, for example, a display unit 310 of the mobile terminal device 300 or a display unit 410 of an information processing device 400 to be described below. Here, the display unit 150 displaying the workout information is not limited thereto and any of display units of various devices, such as a head mounted display (HMD), capable of browsing information can be used.

The processing unit 140 performs a user operation received by the reception unit 110 or various processes based on information or the like stored in the storage unit 120. For example, as will be described below, the processing unit 140 may perform a process of specifying similar workout information similar to given workout information (selection workout information). A function of the processing unit 140 can be realized by any of various processors such as a central processing unit (CPU), hardware such as an application specific integrated circuit (ASIC) or a gate array, or a program.

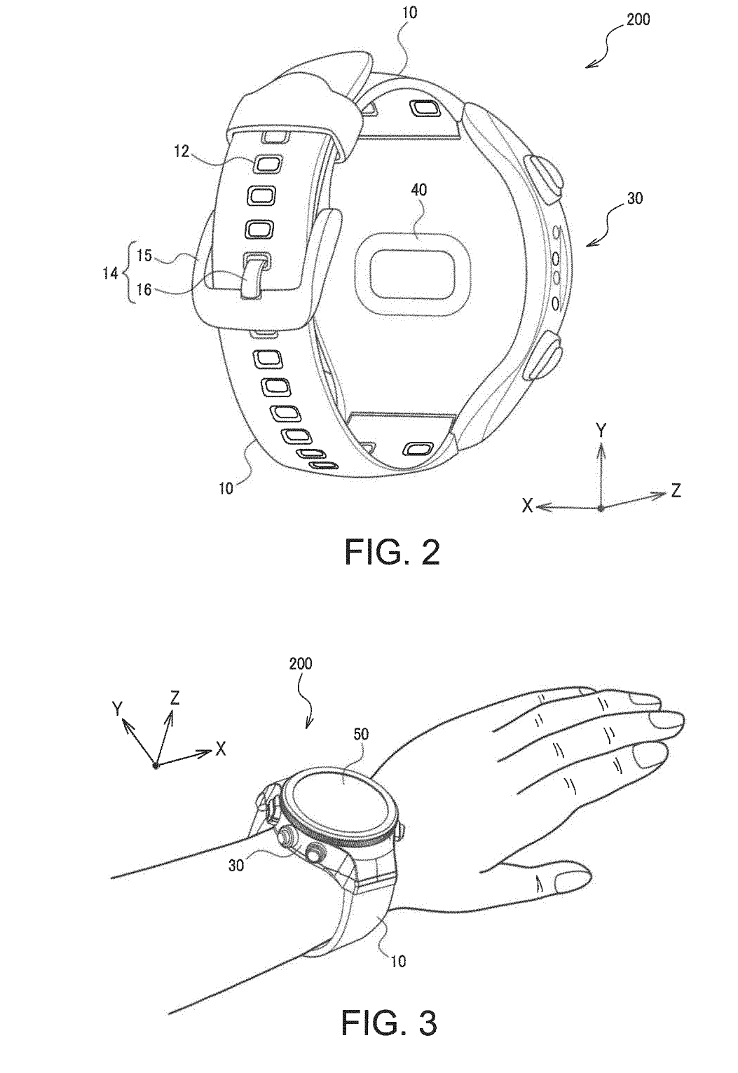

FIGS. 2 and 3 are diagrams illustrating an example of the exterior of a wearable device 200 used to acquire workout information. As illustrated in FIG. 2, the wearable device 200 includes a case unit 30 and a band unit 10 for wearing the case unit 30 on a body (in a narrow sense, a wrist) of the user. In the band unit 10, fitting holes 12 and a buckle 14 are formed. The buckle 14 is configured to include a buckle frame 15 and a locking unit (protrusion rod) 16.

FIG. 2 is a perspective view illustrating the wearable device 200 in a state in which the band unit 10 is fixed using the fitting hole 12 and the locking unit 16 when viewed in a direction on the side of the band unit 10 (a surface of the case unit 30 on a subject side in the wearing state). In the wearable device 200 in FIG. 2, the plurality of fitting holes 12 are formed in the band unit 10. The wearable device 200 is worn on the user by inserting the locking unit 16 of the buckle 14 into any one of the plurality of fitting holes 12. The plurality of fitting holes 12 are formed in a longitudinal direction of the band unit 10, as illustrated in FIG. 2.

A sensor unit 40 (a sensor or a sensor module) that senses various kinds of information is installed on the case unit 30 of the wearable device 200. The sensor unit 40 includes, for example, a body motion sensor or a biosensor. The body motion sensor can be realized by an acceleration sensor, a gyro sensor, an atmosphere sensor, a geomagnetic sensor, a position sensor such as a global navigation satellite system (GNSS) receiver, or the like. The biosensor can be realized by a pulse wave sensor, an arterial oxygen saturation sensor, a temperature sensor, or the like. The sensor unit 40 may include both the body motion sensor and the biosensor or may include one of the body motion sensor and the biosensor. The body motion sensor and the biosensor may be one of the foregoing sensors or a combination of the plurality of sensors. The sensor unit 40 may include a sensor other than the body motion sensor or the acceleration sensor. For example, the sensor unit 40 may include an environment sensor sensing a surrounding environment, for example, an atmosphere sensor, an illuminance sensor, an ultraviolet sensor, a humidity sensor, and a temperature sensor.

FIG. 2 illustrates an example in which a biosensor (in particular, a pulse wave sensor) is assumed and the sensor unit 40 is installed on a surface of the case unit 30 on the subject side when the wearable device 200 is worn. However, the position at which the sensor included in the sensor unit 40 is installed is not limited to the example of FIG. 2. For example, the body motion sensor may be installed inside the case unit 30 (in particular, on a sensor substrate included in the case unit 30).

FIG. 3 is a diagram illustrating the wearable device 200 in state in which the wearable device 200 is worn by the user when viewed on the display surface side of a display unit 50. As understood from FIG. 3, the wearable device 200 according to the embodiment includes the display unit 50 at a position equivalent to a text plate of a normal wristwatch or a position at which numerals or icons can be viewed. In the state in which the wearable device 200 is worn, the surface of the case unit 30 on the side illustrated in FIG. 2 is adhered to the subject and the display unit 50 is located at a position at which the user can easily see the display unit 50.

In FIGS. 2 and 3, a coordinate system is set using the case unit 30 of the wearable device 200 as a reference and a direction intersecting the display surface of the display unit 50 and oriented from the rear surface to the front surface of the display unit 50 when the display surface side of the display unit 50 is defined as the front surface is a Z axis positive direction. Alternatively, a direction oriented from the sensor unit 40 (in a narrow sense, the pulse wave sensor illustrated in FIG. 2) to the display unit 50 or a direction away from the case unit 30 in a normal direction of the display surface of the display unit 50 may be defined as a Z axis positive direction. In the state in which the wearable device 200 is worn on the subject, the Z axis positive direction is equivalent to a direction oriented from the subject to the case unit 30. Two axes perpendicular to the Z axis are XY axes. In particular, a direction in which the band unit 10 is mounted on the case unit 30 is set as the Y axis.

The example of the device held on an arm (wrist) of the user by the band unit 10 has been described as the wearable device 200 with reference to FIGS. 2 and 3. However, the shape or the wearing part of the wearable device 200 is not limited thereto. For example, the wearable device 200 may be a device worn on another part of the user such as an ankle by the band unit 10 or may be an HMD or the like.

When the wearable device 200 including the sensor unit 40 is worn and a workout is executed, workout information corresponding to the workout is acquired.

Here, the workout information includes at least one piece of information among an exercise distance, an exercise time, and an exercise intensity. The exercise distance is information indicating a distance by which the user has moved in a workout. For example, the exercise distance is obtained in accordance with displacement of positional information acquired using a GPS receiver or the like. The exercise time is information indicating a time in which a workout is executed. The exercise distance may be calculated from a movement speed of the user and a time in which a workout is executed. In this case, an exercise distance may be calculated as an accumulation of a section distance obtained from a movement speed for each predetermined section and a section movement time or the exercise distance may be calculated from an average movement speed and a time in which a workout is executed.

The exercise intensity is information indicating intensity of a workout and is information expressed by various measurement values measured by the sensor unit 40 during the workout. The measurement values are information such as a pace, a pitch, consumed calories, and a heart rate (pulse rate), as will be described below. Alternatively, the exercise intensity may be lap information to be described below. A pace or a lap can be calculated from a distance (positional information) and a time. The pitch can be calculated from a movement distance and the number of steps calculated from the body motion sensor (acceleration sensor). The consumed calories can be calculated from a magnitude of a body motion, a heart rate, or the like. The heart rate can be calculated from a sensor signal of the biosensor (a pulse wave sensor: specifically, a photoelectric sensor). Additionally, each measurement value can be known to be acquired based on any of various calculation processes based on various sensor signals. In the embodiment, the various calculation process can be broadly applied. Since any measurement value is changed according to a light exercise or an intense exercise, each measurement value can be used as an index indicating exercise intensity.

The workout information may be a target of a display process or the like using a workout of one time as a unit. The workout of one time is, for example, a time in which a body starts to move and then stops. For example, when a running is executed as a workout, a workout of one time indicates an exercise in which the running starts and ends. Alternatively, a period from a time point at which the user operates the reception unit 110 (operation unit) to start measurement of the workout to a time point at which the user operates the reception unit 110 to end the measurement of the workout may be set as a workout of one time.

Here, when a high-load exercise and a break (a low-load exercise) and repeated a plurality of times as in an interval training or when a workout is executed for a long time, it is natural and is a purpose of the user to consider that an entire interval training or an exercise time including one or more break periods is one workout rather than considering that the workout ends at the break time. When a swimming is executed as a workout, for example, when a user swims a crawl of 50 meters 10 times, the crawl may be considered to be a workout of 10 times and it is meaningful to consider that the swimming of 500 meters is a workout of one time. In this way, the definition of the “workout of one time” is preferably determined according to not only whether the workout is simple but also an intention of the workout or the like.

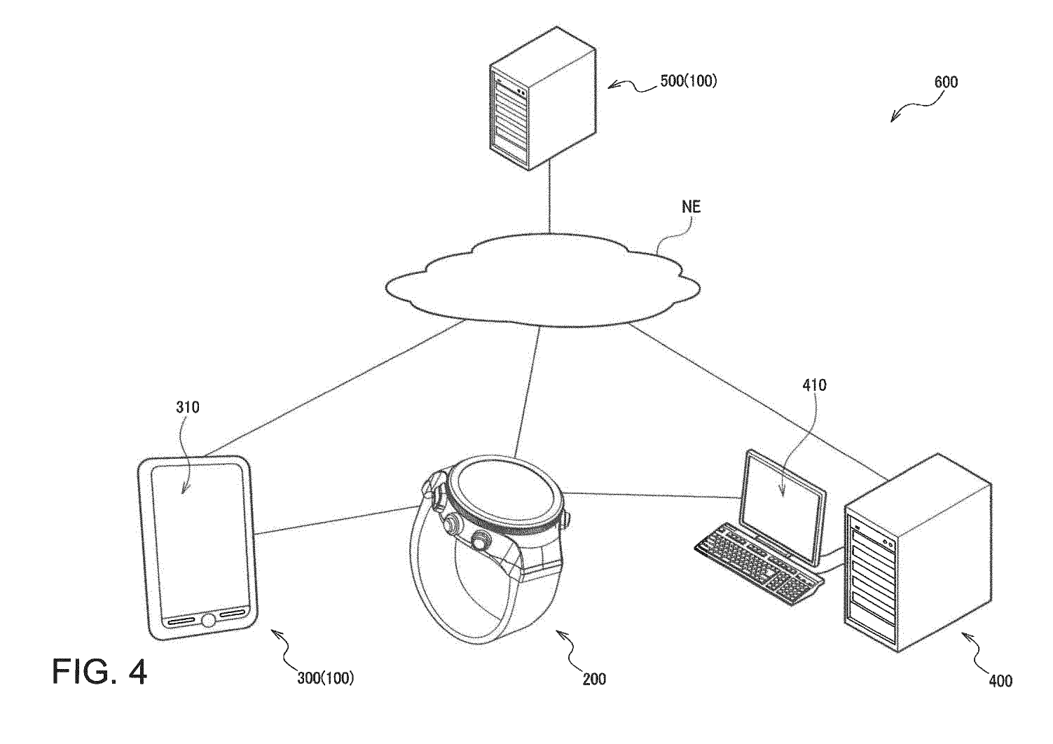

FIG. 4 is a diagram illustrating an example of a system 600 including the workout information display system 100 according to the embodiment. As illustrated in FIG. 4, the system 600 includes the wearable device 200, the mobile terminal device 300 (for example, a smartphone), the information processing device 400 (for example, a PC), and a server system 500. Each of the wearable device 200, the mobile terminal device 300, the information processing device 400 and the server system 500 includes a hardware processor or integrated circuit, and a storage medium. The hardware processor corresponds to a central processing unit (CPU) or equivalents thereof. The integrated circuit corresponds to ASIC circuits, or FPGA circuits, or equivalents thereof. The storage medium corresponds to volatile memory such as random access memory (RAM) or equivalents thereof, or non-volatile memory such as flash memory, hard disks, or equivalents thereof. Each of the wearable device 200, the mobile terminal device 300, the information processing device 400 and the server system 500 includes a transmitter/receiver for transmitting or receiving information over a network. The transmitter/receiver corresponds to devices for communicating over cellular, telephone or internet networks, or equivalents thereof.

The wearable device 200 is configured to be connectable to the mobile terminal device 300 and the information processing device 400. For example, the wearable device 200 may be connected to the mobile terminal device 300 and the information processing device 400 through short-range wireless communication. Here, as the short-range wireless communication, any of various types of communication such as Bluetooth (registered trademark), near field ratio communication (NFC), ANT+, and the like can be applied. Alternatively, the wearable device 200 maybe wired-connected to the mobile terminal device 300 or the information processing device 400 via a cradle or the like.

The mobile terminal device 300 and the information processing device 400 are connected to the server system 500 via a network NE. The network NE is the Internet in a narrow sense. However, the invention is not limited thereto. The network NE can be realized by a wide area network (WAN) or a local area network (LAN) and can be either a wired network or a wireless network.

In this configuration, the wearable device 200 can upload information to the server system 500 via the mobile terminal device 300 or the information processing device 400. That is, in the server system 500, information measured by the wearable device 200 during a workout can be acquired and stored. As illustrated in FIG. 4, the wearable device 200 may be configured to be connectable to the network NE and the wearable device 200 may directly upload information to the server system 500.

A process of obtaining workout information (an exercise distance, an exercise time, or an exercise intensity) from information detected by the wearable device 200 may be performed by the server system 500. This is because the server system 500 is assumed to be better than the other devices in FIG. 4 in terms of performance of a processor, the capacity of a memory, or the like. Here, the process of obtaining workout information may not be hindered from being performed in the wearable device 200, the mobile terminal device 300, or the information processing device 400. Hereinafter, a display process will be described assuming that workout information is obtained by any device.

The workout information display system 100 according to the embodiment can be realized by any of various devices. For example, the scheme according to the embodiment can be applied to an electronic device including the workout information display system 100. Here, the electronic device is, for example, the mobile terminal device 300. In this case, the reception unit 110 receives an operation on an operation unit (a button, a touch panel, or the like) of the mobile terminal device 300 and the display processing unit 130 displays workout information on the display unit 310 of the mobile terminal device 300. Alternatively, the electronic device may be the information processing device 400. In this case, the reception unit 110 receives an operation on an operation unit (a mouse, a keyboard, or the like) of the information processing device 400 and the display processing unit 130 displays workout information on the display unit 410 of the information processing device 400. In this case, the reception unit 110 is realized as an operation interface that receives operation information generated by operating the operation unit of the electronic device.

In this case, the storage unit 120 may store workout information necessary for the display process among the workout information accumulated in the server system 500. When workout information not stored in the storage unit 120 is a display target, the electronic device (the mobile terminal device 300 or the information processing device 400) may access the server system 500, download insufficient workout information, and perform a process of storing the insufficient workout information in the storage unit 120. When the capacity of the storage unit 120 is sufficient, all of the workout information regarding a target user is not hindered from being stored in the storage unit 120.

Alternatively, the scheme according to the embodiment may be applied to the server system 500 including the workout information display system. The server system 500 according to the embodiment includes the reception unit 110 that receives operation information regarding a user, the storage unit 120 that stores workout information based on a workout executed by the user, and the display processing unit 130 that performs a process of displaying the workout information on the display unit 150. The server system 500 may also include the processing unit 140. In this case, the reception unit 110 is not hindered from receiving an operation on an operation unit of the server system 500 and the display processing unit 130 is not hindered from displaying workout information on a display unit (not illustrated) of the server system. Here, there are many cases in which the server system 500 is managed by a manager, and thus free use of each user is not assumed generally.

That is, in this case, the reception unit 110 of the server system. 500 (the workout information display system. 100) receives an operation on an operation unit of another device and performs the process of displaying workout information on the display unit of the other device. For example, the reception unit 110 receives operation information indicating an operation on an operation unit of the mobile terminal device 300 via the network NE and the display processing unit 130 performs a process of transmitting display screen information generated based on the operation information via the network NE.

As understood from the example of the server system 500, a device including the workout information display system 100, a device in which the operation unit is installed, and a device in which the display unit is installed may be different. For example, when the workout information display system 100 is included in the mobile terminal device 300 (the information processing device 400), at least one of the operation unit and the display unit may be installed on a device other than the mobile terminal device 300 (the information processing device 400). For example, a case in which an HMD is used to display workout information is considered. Since the HMD is mounted on a head, the HMD is preferably miniature and light-weighted and it is difficult to provide a complex operation unit. Accordingly, an embodiment is considered in which the display processing unit 130 generates display screen information through an operation on an operation unit of the mobile terminal device 300 including the workout information display system 100 and a display unit of the HMD performs display according to the display screen information.

The example in which the workout information display system 100 is included in any one of the devices has been described. However, the invention is not limited thereto. The workout information display system 100 according to the embodiment may be realized with a distribution process by a plurality of devices.

In the workout information display system 100 or the like according to the embodiment, by a part or the entirety of the process may be realized by a program. In this case, when a processor such as a CPU executes the program, the workout information display system 100 or the like according to the embodiment is realized. Specifically, the program stored in a nontransitory information storage medium (information storage device) is read and the processor such as a CPU executes the read program. Here, the information storage medium (computer-readable medium) stores a program, data, or the like and a function can be realized by an optical disc (a DVD, a CD, or the like), a hard disk drive (HDD), or a memory (a card memory, a ROM, or the like), or the like. The processor such as a CPU performs various processes according to the embodiment based on the program (data) stored in the information storage medium. That is, a program causing a computer (which is a device including an operation unit, a processing unit, a storage unit, and an output unit) to function as each unit according to the embodiment (a program causing the computer to perform a process of each unit) is stored in the information storage medium.

The workout information display system 100 according to the embodiment may include a processor and a memory. Here, for example, the processor may be realized by hardware in which a function of each unit is individual or may be realized by hardware in which a function of each unit is integrated. For example, the processor can include hardware and the hardware can include at least one of a circuit processing a digital signal and a circuit processing an analog signal. For example, the processor can be configured with one circuit device or a plurality of circuit devices (for example, IC) mounted on a circuit substrate or one circuit element or a plurality of circuit elements (for example, a resistor and a capacitor). The processor may be, for example, a central processing unit (CPU). Here, the processor is not limited to a CPU. Various processors such as a graphics processing unit (GPU) and a digital signal processor (DSP) can also be used. The processor maybe a hardware circuit configured with ASIC. The processor may include an amplifier circuit, a filter circuit, or the like processing an analog signal. The memory may be a semiconductor memory such as an SRAM or a DRAM, may be a register, may be a magnetic storage device such as a hard disk device, or may be an optical storage device such as an optical disc device. For example, the memory stores a computer-readable command. The function of each unit of the workout information display system 100 is realized when the command is executed by the processor. Here, the command may be a command which is a command set that configures a program or may be a command instructing a hardware circuit of the processor to operate.

The workout information display system 100 accumulates and display the workout information, as described above. However, the workout information may be associated with another information. For example, the workout information display system 100 may acquire weather, meals, medical data, or the like from a data server and may accumulate and display the acquired information in association with the workout information or the user having executed the workout. Here, the medical data includes information regarding a health diagnosis result, information regarding medical examination in a medical institute, and medical treatment information. Here, the data server may be the same as the server system 500 or may be a different server. For example, the medical data or the like has high protection importance because of individual information, and thus is assumed to be realized by a dedicated server different from the server system 500 that manages workout information.

### 2. Screen Transition Example

As described above, the workout information display system 100 according to the embodiment displays workout information acquired in a workout. Here, since the workout is continuously executed, the amount of workout information becomes vast over time. For example, for even a user who lightly runs once or several times per week, the number of workouts during about 1 year is several times, several tens of times or 100 times or more. For a user who is at an athlete level, it can be considered that a workout is executed almost every day or several times per day. In a workout of one time, information such as an exercise distance, an exercise time, and an exercise intensity is obtained. Such information (in particular, exercise intensity) maybe one piece of information in the entire workout of one time, but information indicating a chronological change during a workout is also useful to generate workout analysis or a workout plan.

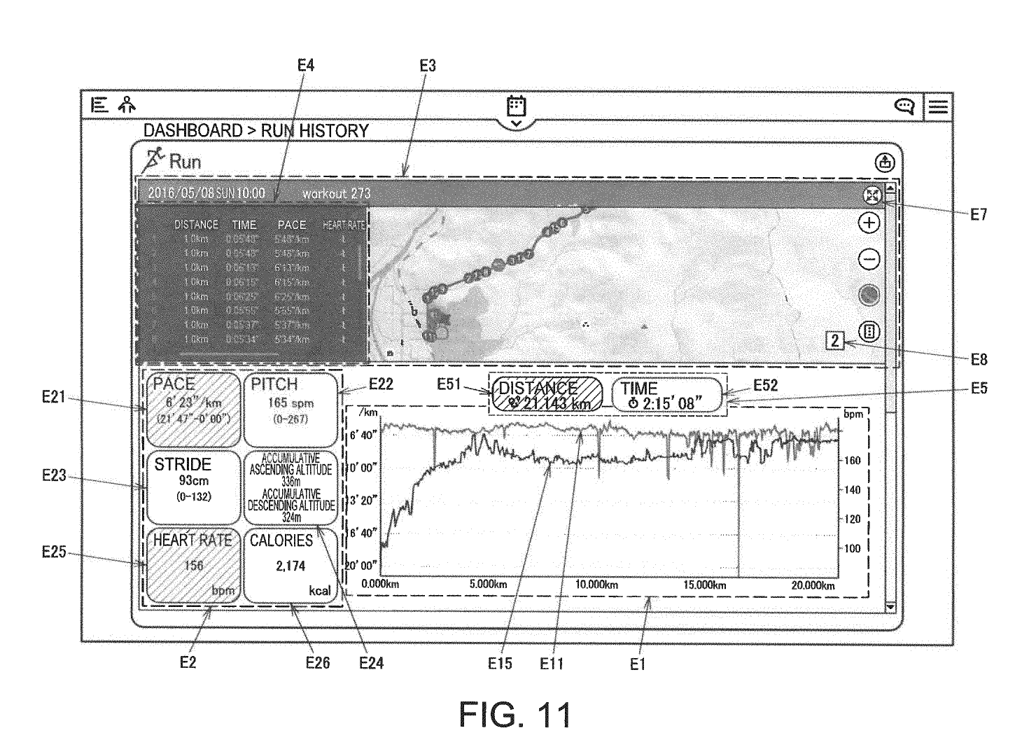

That is, when an amount of information is large as in FIG. 11 to be described below even in workout information corresponding to a workout of one time and the number of workouts becomes large, the entire amount of information becomes vast. In order for a user to appropriately browse desired workout information, it is necessary to carefully examine a scheme of displaying workout information.

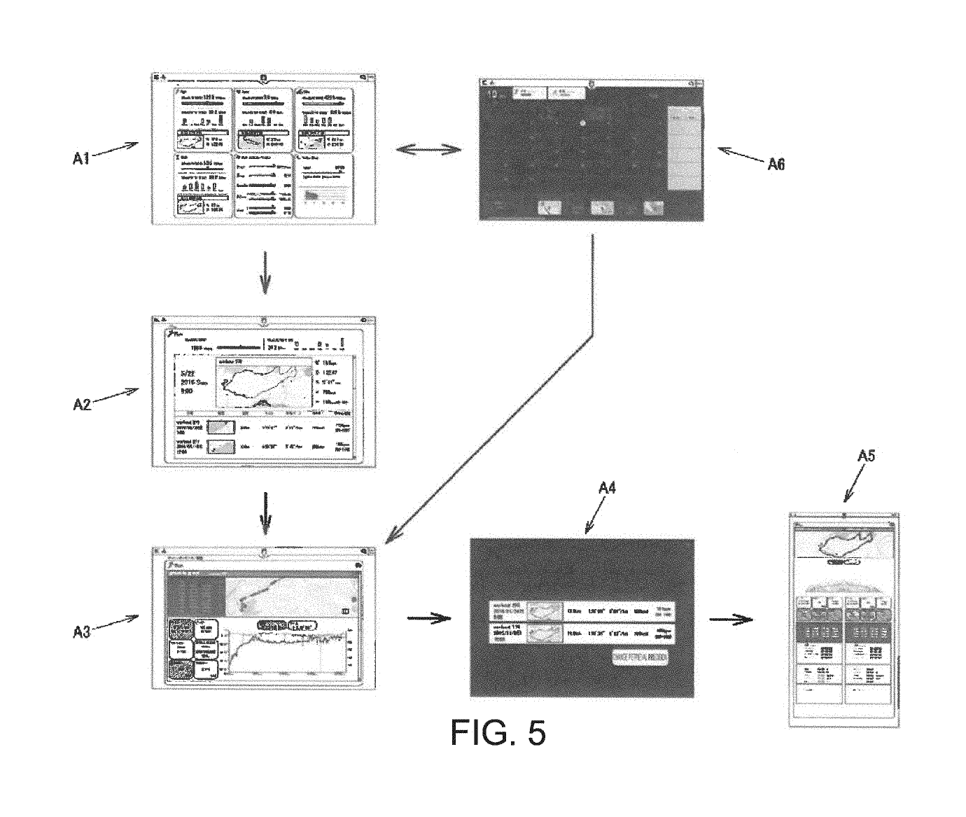

Accordingly, in the embodiment, screen transition illustrated in FIG. 5 is performed. Here, the screen transition in FIG. 5 is merely an example and various modifications can be made, for example, by adding other screens, omitting some of the screens, and adding transition (not illustrated). For example, transition to an immediately previous screen can be realized, by pressing a return button. Alternatively, transition from a display state of any screen to a board screen (A1) may be able to be realized through a specific operation (in a narrow sense, pressing a top page selection button).

First, in the embodiment, the display unit 150 first disposes and displays display objects of first to N-th cards corresponding to first to N-th (where N is an integer equal to or greater than 2) kinds of workouts on a board screen (A1).

Here, the kinds of workouts indicate kinds of methods in which a body moves in the workouts or kinds of sports. For example, the first to N-th kinds of workouts include a running (run), a walking (walk), a bicycle (bike), a swimming (swim). Depending on a running environment, a normal running, a trail run, and a treadmill may be set as different kinds of workouts. Similarly, a walking and a trekking may be different kinds of workouts. Various kinds of sports such as tennis, football, ski, and the like may be set as one kind of workout. Three competitions of a run, a bike, and a swim are executed in a triathlon, but the run, the bike, and the swim may be considered as three kinds of workouts or may be considered as one kind of workout as a whole. Workouts may be classified according to characteristics of workouts executed by a user, such as an exercise in which the user is moving his or her legs, an exercise in which the user takes a ride, and an exercise in which the user is moving in water, and cards may be generated.

The board screen (a dashboard or a dashboard screen) indicates a screen on which various kinds of data are integrated and outlines are summarized to be displayed as a list. The board screen may be a screen displayed at the time of starting display by the display processing unit 130 in a narrow sense. Here, the time of starting the display corresponds to the time of activating a display application by the mobile terminal device 300, the time of access to a display site by a browser of the information processing device 400, or the time of logging in a display site.

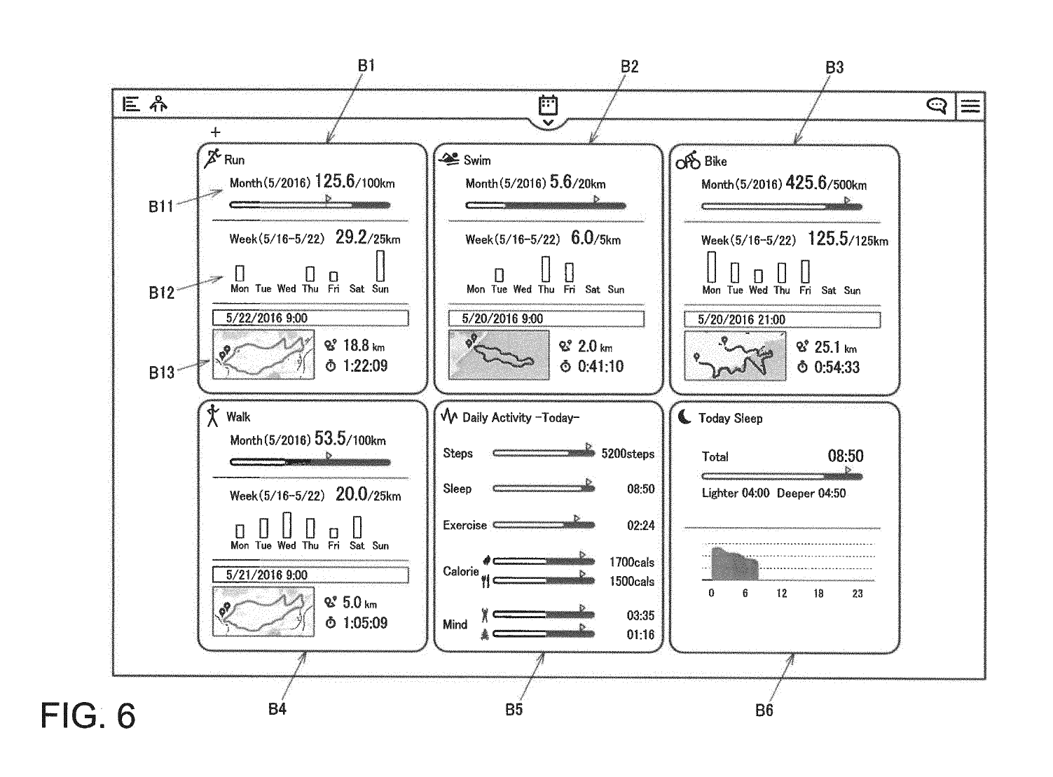

The card (a card object or a card display object) is a unit of an information display region in which information (exercise data) associated with a kind of workout is displayed for each kind of workout (a kind of exercise, a kind of behavior, or a kind of feature) of the user. The shape of the card can be modified variously. For example, as will be described below with reference to FIG. 6, a rectangle (a substantial rectangle with rounded corners) may be used. Since the card is a unit of the information display region on the board screen, a change in display is realized by moving, deleting, and adding a card. A specific display example of the card or details of information included in the card will be described below.

When the reception unit 110 receives a user selection operation of selecting an i-th (where i is an integer in the range of 1≤i≤N) card among the first to N-th cards, the display processing unit 130 displays history information of the i-th kind of workout corresponding to the i-th card. The display of the history information corresponds to display of a display screen of A2 and workout information classified as the i-th kind of workout information is displayed.

In this way, the history information of the kind of workout information corresponding to the selected card can be displayed (screen transition of A1→A2 is performed) through an operation of selecting a card. Since a card is disposed for each kind of workout on the board screen, history information of a desire kind of workout can be easily browsed.

Further, when the reception unit 110 receives an operation of selecting k-th (where k is a integer in the range of 1≤k≤M) workout information among first to M (where M is an integer equal to or greater than 1) pieces of workout information displayed as history information, the display unit 150 (the display processing unit 130) displays detailed information of the selected k-th workout information.

The display of the detailed information corresponds to display of a display screen of A3. Specifically, the display processing unit 130 displays a graph of lap information and measurement values as the detailed information of the workout information. In an example to be described below with reference to FIG. 11, the lap information is displayed in E4 and the graph of the measurement values is displayed in E1.

In this way, the detailed information of the selected workout information can be displayed (screen transition of A2→A3 is performed) through an operation of selecting given workout information from the history information.

In the foregoing flow, information to be displayed can be appropriately transitioned through the operation which can be said to be comprehensible as the selection of the card and the selection of one piece of given workout information from the history information. Since one kind of workout is set with one card on the board screen (A1), an amount of information per kind is restricted, and thus display can be realized in a simple and comprehensible mode. Since a previous history of a target kind of workout is displayed on the display screen (A2) of the history information, an outline of the kind of workout can be ascertained or searching a desired workout can be executed. At this time, when the details of one piece of workout information (information regarding a workout of one time) are displayed, the searching is hindered. Therefore, it is preferable to display one piece of workout information in a simple mode. In other words, display in which it is important to search for (specify) a desired workout may be realized on the display screen of the history information. The details of each piece of workout information may be displayed on the display screen (A3) of the detailed information.

There is a request for desiring to compare the given work information to another workout information (similar workout information) similar to the given workout information. Accordingly, the display processing unit 130 may display a list of similar workout information (A4) through an operation on the display screen (A3) of the detailed information and display a comparison screen (A5) for two pieces of workout information through an operation of selecting a given similar workout information from the list of the similar workout information (A4).

On the board screen (A1), displayed information is switched by selecting a card, that is, specifying the kind of workout. Here, for a workout in which an execution period (for example, August in the last year) is known, it is easy to specify the workout information in many cases when a calendar screen is used. Accordingly, when the calendar screen (A6) is displayed and an operation of selecting given workout information on the calendar screen is displayed, the display processing unit 130 may perform a process of switching the calendar screen to the display screen (A4) of the detailed information. The board screen and the calendar screen are assumed to be mutually switchable.

Hereinafter, specific examples of the board screen (A1), the display screen (A2) of the history information, and the display screen (A3) of the detailed information will be described. Further, the display screens (A4 and A5) of the similar workout information will be described. Finally, the calendar screen (A6) will be described.

### 3. Board Screen (Dashboard Screen)

FIG. 6 illustrates an example of the board screen (A1). In the example of FIG. 6, the display processing unit 130 displays six cards (B1 to B6) as first to N-th cards. Here, the number of cards may be equal to or less than 5 or may be equal to or greater than 7. FIG. 6 illustrates an example in which the number of cards displayed at a time on the display screen is 6. Therefore, when the number of cards is equal to or greater than 7, the display processing unit 130 may perform scroll display.

Here, the display screen indicates a display target region of an image including workout information in the workout information display system 100 according to the embodiment. When a plurality of windows can be disposed on a display unit as in a PC, the display screen according to the embodiment corresponds to one window. In a case of a device such as a smartphone that displays an application which is being executed on an entire display unit, the display screen is the same region as a display region of the display unit.

In the example of FIG. 6, B1 to B4 indicate cards corresponding to kinds of workouts (kinds of exercises). B1, B2, B3, and B4 correspond to a run, a swim, a bike, and a walk, respectively.

In this way, in the scheme according to the embodiment, display of the board screen is performed using the cards corresponding to the kinds of workouts. Since the card is used for each kind of workout as in the card of the run and the card of the walk, information regarding each kind of workout can be presented to a user in a simple and plain display form.

In particular, devices (the wearable device 200) corresponding to multi-sports are much in demand recent years. Since such devices can be used commonly by different and diverse users who execute sports (in a broad sense, kinds of workouts), there are considerable advantages for either the users or device makers. In this case, by displaying workout information for each kind of workout, display can be realized in an easily usable (high versatile) form for many users. For example, display or the like in which kinds of sports in which a target user is interest are narrowed down can be realized. A case in which one user executes diverse sports can be sufficiently considered. Even in this case, by performing display of the workout information for each kind of workout, it is possible to present an outcome in each sports to the user comprehensibly.

The cards of the embodiment may include cards according to the kinds of workouts and cards according to different kinds of cards are not hindered from being included. For example, the cards may include cards according to kinds of information. Here, the kinds of information mean specific parameters. For example, the kinds of information may be information regarding consumed calories, a physical fitness index, the number of steps, a weight, a height, BMI, a body fat percentage, a calorie balance, a mind balance (a balance between a stress state and a relax state), and the like.

B5 and B6 in FIG. 6 indicate cards corresponding to kinds of information. B5 corresponds to an outline of plurality of pieces of information (the number of steps, a sleep, an exercise time, consumed calories, and a mind balance) and B6 corresponds to a sleep.

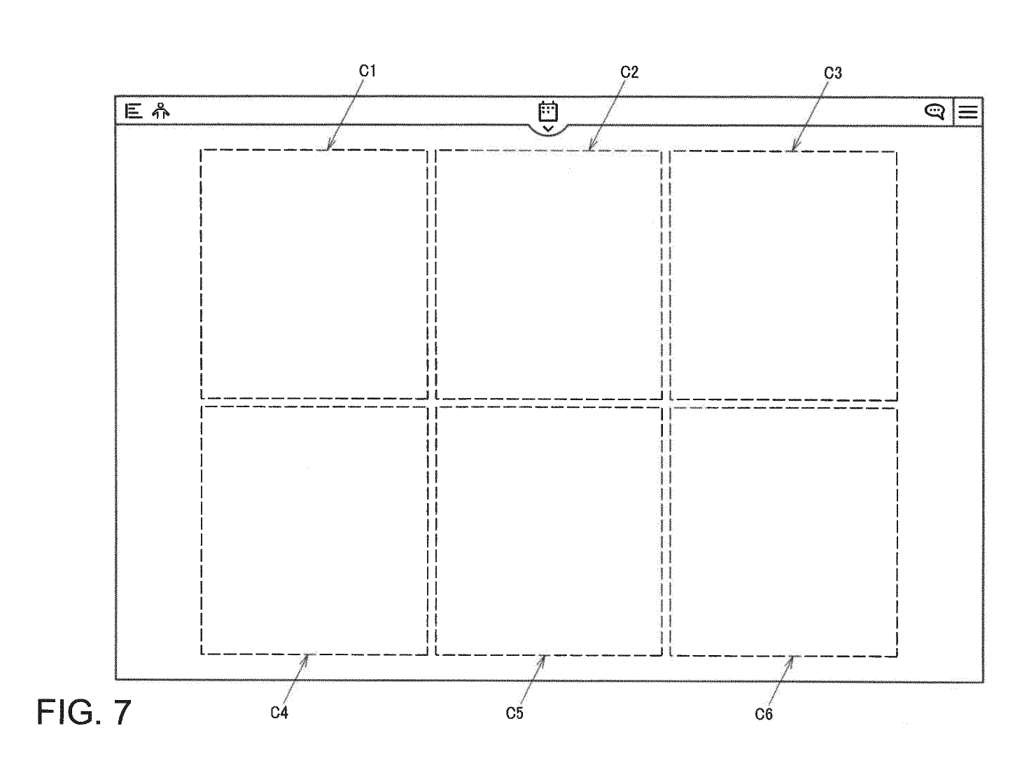

On the board screen, regions in which the cards can be disposed may be set in advance. For example, as illustrated in FIG. 7, disposition regions C1 to C6 of six spots may be set on the board screen. Here, the number of disposition regions is not limited to six and is determined according to the maximum number of cards which can be displayed on the board screen. Scroll display can be realized on the board screen. The number of disposition regions (the maximum number of cards) may not necessarily depend on a resolution of the display unit (the display screen). The number of disposition regions set per row (per column) may be fixed or may be changed according to the resolution of the display unit (the display screen). For example, setting can be realized in such a manner that scroll in the vertical direction of the board screen is allowed and scroll in the horizontal direction is not allowed. In this case, the number of disposition regions per row (the number of disposition regions arranged in the horizontal direction) depends on the resolution in the horizontal direction of the display screen. As an example of the embodiment, the sizes of the plurality of cards displayed on the board screen are the same, but the invention is not limited thereto. Card with different sizes can be displayed. In this case, when the size (the vertical width and the horizontal width) of the smallest card is set as a reference and the cards with lengths of integer multiples are displayed in at least one of the vertical and horizontal directions, the cards can be easily arranged despite different sizes of the cards. Therefore, it is possible to appropriately set and change an amount of necessary display information in accordance with the kinds of cards while maintaining a list property of the board screen.

Each card is disposed in one of the disposition regions. In the example of FIG. 6, the cards B1 to B6 are disposed in the disposition regions C1 to C6, respectively. The cards are not allowed to be disposed out of the disposition regions. In this way, the cards can be arranged to be disposed at predetermined positions. In the example of FIG. 6, the positions (upper ends and lower ends) of the cards B1 to B3 in the vertical direction are arranged and the positions (left ends and right ends) of the cards B1 to B4 (B2 and B5 or B3 and B6) in the horizontal direction are arranged. That is, since the cards can be orderly disposed, information can be presented to the user comprehensibly.

For each card which can be displayed on the board screen, a display position can be changed or display/non-display can be set. For example, a display position of a given card can be changed through a drag operation on the card. At this time, when another card is already disposed at a movement destination, the display positions of the cards may be switched. More specifically, when an operation of moving a given card to another disposition region is executed, the display processing unit 130 may perform a process of exchanging disposition regions at which the two cards are displayed.

By setting the disposition regions, it is easy to also dispose the cards again. For example, the display processing unit 130 may display the cards so that the cards are aligned to the top and the left. Specifically, a card display priority of the disposition region C1 is highest and is set to be lowered in order of C2→C3→C4→C5→C6. Even when there are cards subsequent to C7, the display priority is similarly set. Here, the example in which the cards are disposed to be aligned to the top and the left has been described, but those skilled in the art can easily understand that various modifications can be made.

When a card disposed in a given disposition region Cx (where x is an integer equal to or greater than 1) is set to non-display and there are cards in disposition regions subsequent to Cx+1, each card may be aligned to the immediately previous display position one by one. Specifically, a card displayed in Cx+1 is displayed in Cx and a card displayed in Cx+2 is displayed in Cx+1. The same applies to cards displayed in disposition regions subsequent to Cx+3. For example, when the card (B3) of the bike in the state of FIG. 6 is set to non-display, the display position of the card (B4) of the walk is changed to C3. Similarly, the display position of the card of B5 is changed to C4 and the display position of the card of B6 is changed to C5. When cards are not disposed after the seventh disposition region, any card is not displayed in the disposition region of C6.

In a case in which a card is newly added to the disposition region Cx in which the card has already been displayed, the display position of each card may be shifted backward one by one. Specifically, the card which has been displayed in Cx is displayed in Cx+1 and the card which has been displayed in Cx+1 is displayed in Cx+2.

Even when the above-described movement of the cards is performed, re-disposition of the cards aligned to the left may be used. Specifically, when an operation of moving a given card to a different disposition region, the display processing unit 130 displays the given card in a designated disposition region and disposes another card again so that the card is aligned to the top left. For example, when the card (B1) of the run is moved to the disposition region of C4 in the state of FIG. 6, the card of B1 is displayed in C4 and the cards of B2 to B4 are disposed again since C1 is empty. Specifically, the card (B2) of the swim is moved to C1, the card (B3) of the bike is moved to C2, and the card (B3) of the walk is moved to C3. In such a scheme, the movement of the cards can be realized. Addition of a card to the disposition region Cx may be realized by a step by step operation. For example, an addition card may necessarily be added to the disposition region which is the rearmost region (a disposition region in which no card is displayed and which has the highest priority). Thereafter, the addition card may be moved to the disposition region Cx through an operation of moving the card.

When the order of the cards is shifted, a function of fixing a card not desired to be moved may be installed. For example, when the card displayed in the disposition region Cx is fixed, the display processing unit 130 may exclude the disposition region Cx in the order shifting process for the cards through movement, non-display, and addition of the cards. Specifically, the foregoing process is executed setting C1 to Cx−1 and the regions subsequent to Cx+1 as targets. In this way, other cards can be appropriately shifted while fixing the card displayed in the disposition region Cx. Display indicating the fact that the cards (for example, display of a pin icon) can be appropriately shifted may be performed in the fixed card.

When the reception unit 110 (a communication interface or a reception unit) receives workout information of the kinds of workouts not displayed on the board screen, the cards corresponding to the kinds of workouts included in the workout information may be displayed on the board screen without receiving an operation or setting from the user.

When there is no workout information transmitted to the workout information display system 100 by the user, the cards corresponding to kinds of information, for example, the cards B5 and B6 in FIG. 6, maybe displayed on the board screen. When the user transmits the workout information to the workout information display system 100 for the first time, the cards corresponding to the kinds of workouts included in the workout information may be displayed on the board screen without receiving an operation or setting from the user.

The display unit 150 (the display processing unit 130) displays summary information of the corresponding kind of workout in regard to the card (each of the first to N-th cards). In this way, an outline of the kind of workout corresponding to the card can be displayed in regard to each card, and thus the simple and comprehensible information can be presented to the user. It is not necessary to apply the configuration of the invention in the present specification to all of the first to N-th cards. For example, when the display processing unit 130 displays the first to fifth cards on the board screen, a relation of the invention in the specification may be established only in the first to third cards among the first to fifth cards.

Here, the summary information is statistical information for a predetermined period. Then the statistical information for the predetermined period may be at least one of statistical information for each month and statistical information for each week. FIG. 6 illustrates an example in which both statistical information (B11) for one month and statistical information (B12) for one week are displayed in the card (B1) of the run. Here, the statistical information of one of B11 and B12 maybe omitted or statistical information for another period may be added. Here, the predetermined period may not necessarily be continuous. For example, a modification in which statistical information is generated and displayed using data of only Sunday in a given one month is not hindered either.

In FIG. 6, for the statistical information (B11) for one month, a target exercise distance (a target value: 100 km) for one month and a sum value (an actual value: 125.6 km) of distances by which a user actually takes an exercise for a target period are displayed as numerical values. Along with display of the numerical values, an achievement ratio (the degree of achievement) of the actual value to the target value is displayed with a graph.

For the statistical information (B12) for one week, a target exercise distance (a target value: 25 km) for one week and a sum value (an actual value: 29.2 km) of distances by which a user actually takes an exercise for a target period are displayed as numerical values. Along with display of the numerical values, an actual value for every day of the week (every day) is displayed with a graph.

FIG. 6 illustrates an example in which one bar graph is displayed in B11 and a total of seven bar graphs for every day of the week (every day) are displayed in B12. Here, a modification can be made in which a ratio of the actual value to the target value can be displayed with one bar graph in B12 or actual values of everyday (or each week) are displayed with a plurality of bar graphs in B11. Those skilled in the art can understand that a graph other than the bar graph may be used in the graph display of the statistical information and numerals may be displayed.

The display unit 150 (the display processing unit 130) displays map information for a most recent workout among the corresponding kinds of workouts in the cards (the first to N-th cards). FIG. 6 illustrates map information (B13) of the most recent workout (a run or a running) in the card (B1) of the run. Here, it is not necessary to apply the configuration of the invention in the present specification to all of the first to N-th cards. That is, the display processing unit 130 is not hindered from setting some of the cards as targets and displaying the map information.

In this way, at least the map information among various kinds of pieces of most recent workout information can be displayed on the board screen. As illustrated in FIG. 6, another information (a date, an exercise distance, and an exercise time) included in the most recent workout information may also be displayed. As illustrated above, the board screen is a screen assumed to be first browsed by the user. Since the user attempts to browse the workout information to know a result of a workout after the user performs the workout, the user is considered to be strongly interested in the most recent workout. That is, by displaying the most recent workout information, it is possible to present appropriate information to the user. At this time, since the map information is information which is easier to understand intuitively than numerical values, the map information can be said to be information appropriate for display on the board screen on which the outline is presented.

Here, the map information may be information regarding a map corresponding to a place in which a workout is performed or may be information in which a movement route of a workout is superimposed on a map.

In the example of FIG. 6, statistical information and map information are also displayed as summary information in the other kinds of workouts, that is, the swim (B2), the bike (B3), and the walk (B4), as in the run.

As described above, the card is generated for each kind of workout (kind of exercise) and data (exercise data: an exercise distance, an exercise time, a date, weather, a map, a route, the degree of target achievement, the number of steps, and the like) included in the workout information in each card is displayed on the board screen. However, the number of cards corresponding to one kind of workout is not limited to 1. For example, workout information (exercise data) acquired over different unit periods in the same kind of workout can also be displayed with different cards. For example, apart from the card illustrated in B1, a card in which exercise data of each day run is displayed or a card in which exercise data of a run for a period such as three months or half a year is displayed may be used.

When an operation (for example, a click operation or a tap operation) is performed on at least a partial region of each card, the display processing unit 130 transitions the screen to the screen (A2) on which the history information of the kind of workout corresponding to the operated card is displayed.

### 4. Display Screen of History Information

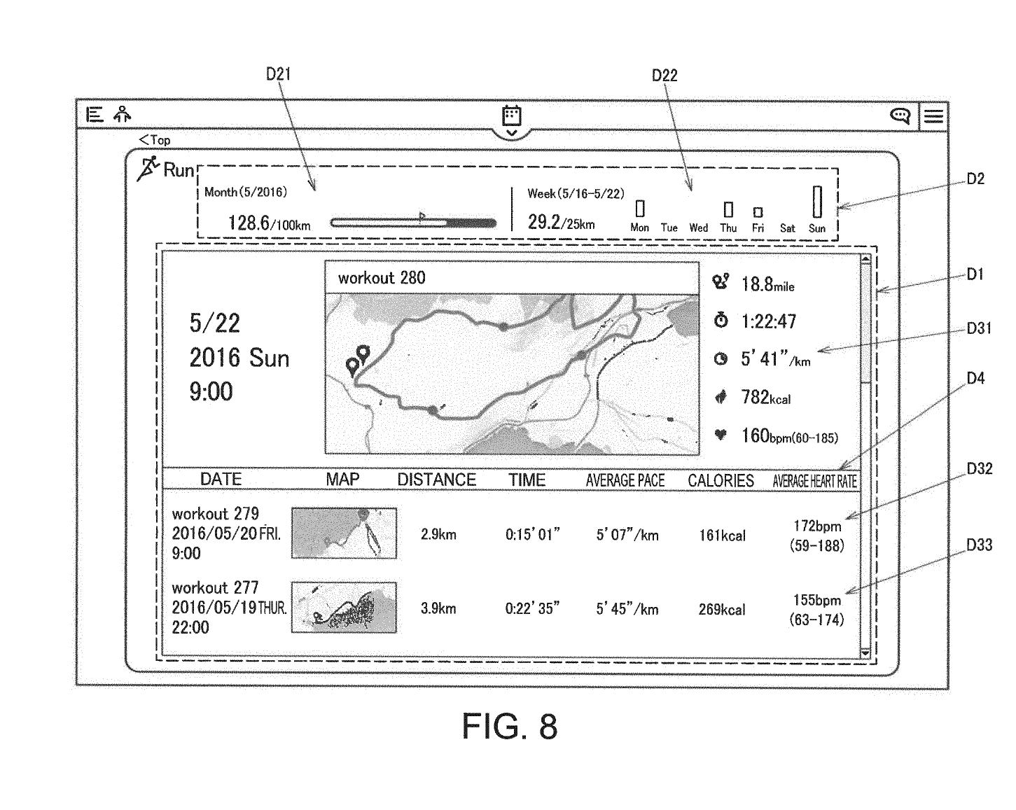

FIG. 8 illustrates a specific example of the display screen (A2) of the history information. Hereinafter, the display screen of the history information when the kind of workout is a run will be described. However, the same display can also be, of course, realized for other kinds of workouts.

The display unit 150 (the display processing unit 130) displays first to M-th (where M is an integer equal to or greater than 1) pieces of workout information indicating first to M-th workouts executed by the user as history information. In this way, unlike the board screen (card) illustrated in FIG. 6, information regarding a workout other than a most recent workout is also a display target. Therefore, it is possible to perform display for confirming a history of workouts executed until now by the user in regard to desired kinds of workouts. Further, by displaying the first to M-th pieces of workout information, it is easy to select any one of the pieces of work information and it is also easy to transition the display screen to the display screen of the detailed information of each workout (A2→A3).

More specifically, the display unit 150 (the display processing unit 130) displays the first to M-th pieces of workout information indicating the first to M-th workouts executed by the user as the history information in a first display region (D1) of the display screen and displays first statistical information calculated based on workout information corresponding to a first period in a second display region (D2) of the display screen.

In FIG. 8, a horizontally long rectangular region disposed on the upper side of the display screen is the second display region (D2) and a rectangular region on the lower side of the second display region is set as the first display region (D1), but the disposition of each display region can be modified variously.

Here, the history information (the first to M-th pieces of workout information) may include map information, as illustrated in FIG. 8. Since the map information is information which is easy to intuitively understand, the workout information by the user can be easily selected.

The display unit 150 may display most recent workout information among the first to M-th pieces of workout information displayed as the history information with a size larger than the other workout information. In the example of FIG. 8, the size of a region in which the most recent workout information (D31) is displayed is greater in the vertical direction than the size of a region in which the other workout information (D32 and D33) is displayed. As described above, it is estimated that the user is strongly interested in the most recent workout information when the user browses the workout information. Accordingly, by displaying the most recent workout information with a large size, it is possible to present important information in a readable display form.

In FIG. 8, the workout information includes a date, map information, an exercise distance, an exercise time, apace (average pace), consumed calories, a heart rate (average heart rate) as a display target. Here, another information (a lap, an altitude, an altitude difference, and the like) may be displayed as the workout information serving as the history information.

In FIG. 8, the most recent workout information and the other workout information are different in a display size, but information amounts thereof are the same. Here, in view of high importance of the most recent workout information, an information amount of the most recent workout information, that is, the display region may be greater than the information amount of the other workout information, that is, the display region. For example, on a display screen of the history information, information regarding a lap which is not a display target in the other workout information may be set as a display target in the most recent workout information.

Since the workout is continuously executed, the number (value of M) of pieces of workout information which are display targets as the history information is assumed to be a large number to some extent. Therefore, it is not easy to display all of the first to M-th pieces of workout information at a time. Accordingly, when the reception unit 110 receives a scroll operation by the user on the first display region, the display unit 150 (the display processing unit 130) may change the workout information displayed in the first display region. Here, the scroll operation can be realized as a mouse wheel rotation operation, a scroll bar dragging operation, a slide operation, or the like.

In this way, the workout information displayed in the first display region can be changed (scrolled). Therefore, even when the number of pieces of workout information increases, the history information can be appropriately browsed. In particular, as illustrated in FIG. 8, when the map information is included in the workout information, it is necessary to set the display size of one piece of workout information to be large to the degree that the map can be browsed, and thus it is useful to browse the history information through the scroll operation.

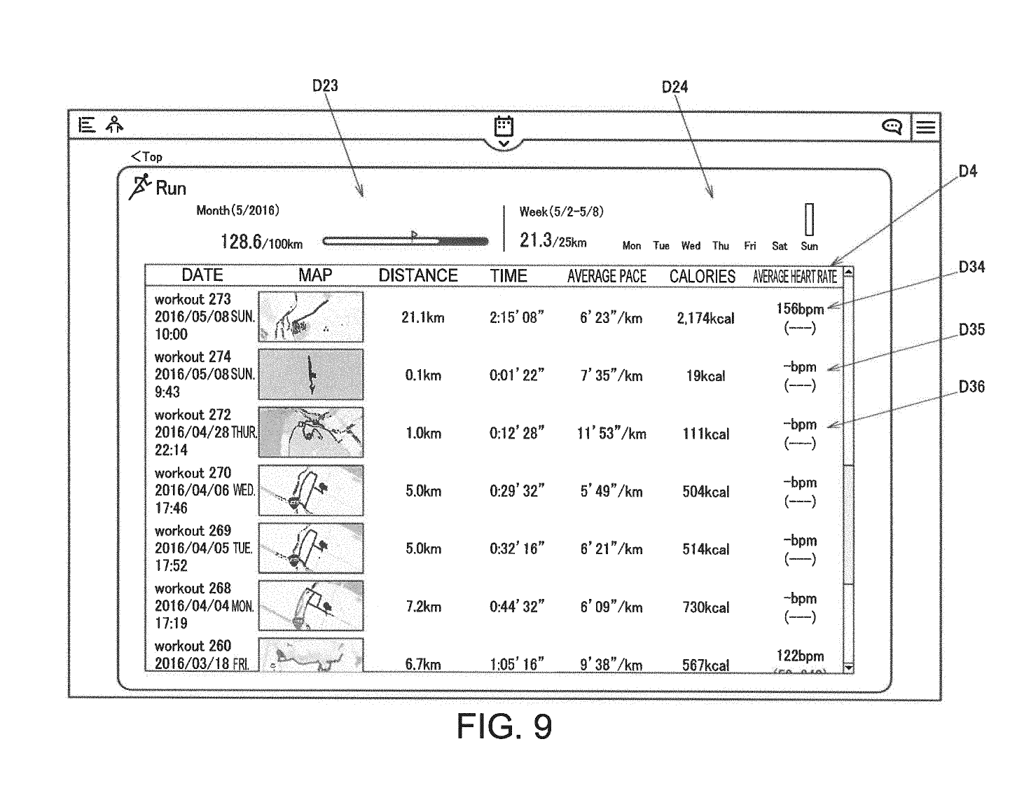

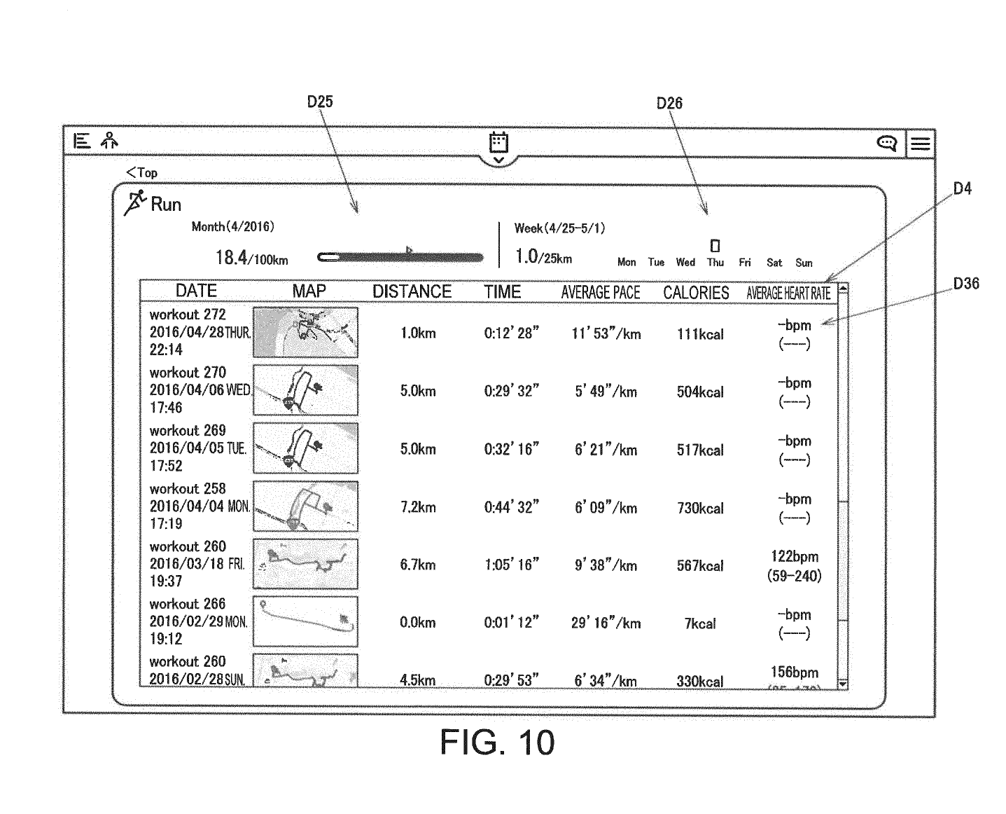

FIGS. 9 and 10 are diagrams illustrating examples of the display screen when a scroll operation is executed on the first display region. In the example of FIGS. 8 to 10, a list of the first to M-th pieces of workout information is chronologically displayed. The workout information acquired at a newer timing is displayed upwardly and the workout information acquired at an older timing is displayed downwardly.

By executing a scroll operation of moving downwardly from the state of FIG. 8, the pieces of workout information D31 to D33 in FIG. 8 deviate from the display target and chronologically earlier workout information D34 is displayed at the uppermost position of the first display region, as illustrated in FIG. 9. By executing a scroll operation further moving downwardly from the state of FIG. 9, the pieces of workout information D34 and D35 also deviate from the display target and chronologically earlier workout information D36 is displayed at the uppermost position of the first display region, as illustrated in FIG. 10.

Here, it is not necessary to execute the scrolling in units of workout information (one row unit of the list) and continuous scrolling (smooth scrolling) maybe executed. For example, a part of given workout information may be allowed to be cut out and displayed on the topmost of the first display region.

To clarify meanings of numerical values displayed as each workout information in FIGS. 9 and 10, an item display region (D4) representing items is set to be necessarily be displayed. Specifically, the item display region displayed near the middle of the first display region in FIG. 8 is moved upward in the first display region through a downward scroll operation. However, after the item display region arrives at the upper end of the first display region the item display region is fixed at the upper end without being in a non-display state (FIGS. 9 and 10). Alternatively, when first workout information is displayed in the first display region, the item display region is disposed between the first workout information and second workout information. When the first workout information is not displayed in the first display region, the item display region can also be disposed at the upper end (upper side) of the first display region. In such a configuration, the workout information (in this case, the most recent workout information, that is, the first workout information) in which the user is most interested in the kinds of workouts is displayed in a large display region so that the workout information can be ascertained at first sight. When the user is interested in the past workout information (history workout information), the history information can be displayed in the first display region as much as possible while executing scrolling. Therefore, the user can easily ascertain the workout information of the kinds of workout.

When the first workout information (for example, the most recent workout information) is displayed, the first workout information is displayed under the second region. When the first workout information (for example, the most recent workout information) is not displayed, the item display region is displayed under the second region. Thus, it is possible to obtain the same advantages.

The history information may be configured such that the history information can be rearranged in the items in the item display region. In such a configuration, the user can browse the history information of the kind of workout from the viewpoint of a date, a place, a distance, a time, and an average pace, calories, an average heart rate, and the like.

In the first display region, the most recent workout information of the kind of workout is displayed, and also the workout information designated by the user may be displayed in the first display region at the time of displaying the history information. For example, workout information of which performance is the highest among the history information of the kinds of workouts may be displayed in the first display region. Alternatively, workout information, when the performance is low such as a slump period, may be displayed in the first display region. Alternatively, the user may set a workout desired to be set as a benchmark as a designated workout and the designated workout may be displayed in the first display region.

Statistical information (each of the first statistical information and second statistical information to be described below) displayed in the second display region (D2) is at least one of statistical information for each month and statistical information for each week. In the example of FIG. 8, both statistical information (D21) for each month and the statistical information (D22) for each week are displayed as the statistical information, but one of the pieces of statistical information may be omitted. In FIG. 8, one bar graph is illustrated as the statistical information for each month and seven bar graphs of every day of the week are illustrated as the statistical information for each week. The specific display of each piece of statistical information can be modified variously as in the display of the statistical information in the cards. The display example of the statistical information in the cards has been described above using B11 and B12 of FIG. 6.

In consideration of a difference of an exercise habit of each user, both the statistical information for each month and the statistical information for each week are preferably displayed. This is because a user who lightly executes a workout as a hobby generally executes a workout only about once per week. In this case, the statistical information for each week is not so useful and the statistical information is used to determine whether a target is achieved monthly. On the other hand, when the user is an athlete, an execution frequency of a workout is high and enhancement of a bodily function can also be considered to be achieved while changing a load of the workout during one week. Accordingly, when an athlete is set as a target, the statistical information for each week can be said to be important as well. That is, by displaying both the statistical information for each month and the statistical information for each week, the display can be performed in a form which users having different exercise habits can generally use.

As described above, in the embodiment, the first to M-th pieces of workout information and statistical information for a predetermined period can be displayed. In this way, information regarding each workout of one time and statistical information for a predetermined period can be presented to the user in a comprehensible display form.

At this time, the first and second display regions may be arranged in one display screen (for example, a window). In this way, since both the first to M-th pieces of workout information and the statistical information are displayed in one screen, it is easy for the user to easily browse the information.

When it is considered that the first to M-th pieces of workout information (the history information) and the statistical information are simultaneously browsed, the foregoing scroll operation is not executed setting the entire display screen illustrated in FIG. 8 as a target, but may be executed setting only the first display region as a target. In this way, a change in the display stays in the first display region and the positions of the first and second display regions in the display screen are fixed. In other words, the second display region is not excluded from the display target through the scroll operation. That is, regardless of a situation of the scroll operation, the statistical information and the first to M-th pieces of workout information (in a narrow sense, some of the pieces of workout information) can be in one display screen.

As described above, by displaying the first to M-th pieces of workout information, it is possible to browse not only the most recent workout information but also past workout information. Here, necessity for browsing past information is not limited to information in which a workout of one time is set as a unit. For example, it is considered that there is a strong request for referring to not only the statistical information for most recent one month (or one week) but also the statistical information for the last month (last week) or the further past statistical information. In this way, this is because information, for example, how data changes on a monthly basis (on a weekly basis) can be presented to the user.

In the scheme of the related art, to change a calculation target period of the statistical information, it is necessary to execute an explicit switching operation. For example, when the statistical information of the last month (the last week) is browsed, it is necessary to press a button and transition a screen. However, then, an operation load of the user is large and an interface which is easily used may not be realized.

Accordingly, in the embodiment, a calculation target period of the statistical information is switched according to a display state of the workout information in the first display region. Specifically, when at least one piece of workout information corresponding to a second period different from the first period is displayed in the first display region based on a user operation received by the reception unit 110, the display unit 150 (the display processing unit 130) displays the second statistical information calculated based on the workout information corresponding to the second period in the second display region. Here, the workout corresponding to the first period can include most recent workout information.

In this way, the statistical information displayed in the second display region is switched in association with display content of the history information. The workout information displayed in the first display region is considered to be a browsing target of the user. That is, by associating the statistical information with the display content of the first display region, the calculation target period of the statistical information can be automatically switched to a period in which the user has an interest.

In this way, since a timing at which the workout information displayed in the first display region is acquired corresponds to the calculation target period of the statistical information displayed in the second display region, it is possible to unify the displayed information temporally in consideration of the entire display screen.

For example, as illustrated in FIGS. 8 to 10, when the workout information corresponding to the second period is displayed in a predetermined region of the first display region, the display unit 150 displays the second statistical information in the second display region. Here, the predetermined region is an upper region of the first display region and a predetermined period is set based on a timing of the workout information displayed in the uppermost portion among the plurality of pieces of workout information displayed in the first display region. Here, the predetermined region can be modified variously.

In the state of FIG. 8, the most recent workout information (D31) displayed to be relatively large is displayed in the uppermost portion of the first display region. Accordingly, the predetermined period is set based on a timing (2016/05/22) at which a workout corresponding to the most recent workout information is executed. Here, as the predetermined period, an example in which one month is a month from a first day to the last day of each month and one week is from Monday to Sunday is indicated. Accordingly, as the statistical information in the state of FIG. 8, the statistical information (D21) of May (5/1 to 5/31) in 2016 and the statistical information (D22) of 5/16 to 5/22 are displayed. Here, the starting day and ending day of the predetermined period (one month or one week) can be modified variously.

In the state of FIG. 9, the workout information (D34) of a workout executed on 2016/05/08 is displayed in the uppermost portion of the first display region. Accordingly, statistical information in which one month is set as the predetermined period is statistical information (D23=D21) in May, 2016 without a change in the state of FIG. 8. The statistical information in which one week is set as the predetermined period is changed to statistical information (D24) of 5/2 to 5/8.

In the state of FIG. 10, workout information (D36) of a workout executed on 2016/04/28 is displayed in the uppermost portion of the first display region. Accordingly, statistical information in which one month is set as the predetermined period is changed to statistical information (D25) in April, 2016 and statistical information in which one week is set as the predetermined period is changed to statistical information (D26) of 4/25 to 5/1.

A trigger for changing the statistical information is considered to be modified variously. For example, the predetermined region may be set to a middle portion or a lower portion rather than the upper portion of the first display region. In this case, the calculation target period of the statistical information is set based on the workout information displayed in the middle of the first display region and the workout information displayed in the lowermost portion.

Alternatively, in the setting of the calculation target period of the statistical information, it is also possible that the predetermined region is not used. For example, when a plurality of pieces of workout information are displayed in the first display region, a period to which each piece of workout information corresponds is determined. In the example of the statistical information for one month, whether each piece of workout information corresponds to a workout executed in which month is determined. The number of workout information as display targets in each month may be aggregated and a period in which the number of pieces of workout information which are display targets is large may be set as the calculation target period of the statistical information. For example, in the state of FIG. 9, two workouts are executed in May, four workouts are executed in April, and one work is executed in March. Therefore, the statistical information of April in 2016 is the display target.

Additionally, display switching of the second display region associated with the display content of the first display region can be modified variously.

### 5. Display Screen of Detailed Information

### 5.1 Graph Display and Numerical Display of Measurement Values

FIG. 11 illustrates a specific example of the display screen (A3) of the detailed information. As illustrated in FIG. 11, the display unit 150 (the display processing unit 130) displays a graph in which a first axis presents a distance or a time and a second axis represents a measurement value of each measurement item of workout information in the first display region (E1). When there are first to P-th (where P is an integer equal to or larger than 2) measurement items as the measurement items, the display processing unit 130 displays first to P-th icons (also referred to as buttons or parameter buttons) for selecting a measurement value of each of the first to P-th measurement items set on the second axis of the graph in the second display region (E2) and displays the measurement value of the measurement item corresponding to the ion in regard to at least one of the first to P-th icons. The number of icons in which the measurement values are displayed can be modified variously. The display unit 150 may display the first to P-th measurement values of the first to P-th measurement items in regard to the first to P-th icons. Here, it is not necessary to apply the configuration of the invention in the present specification to all of the first to P-th icons. For example, the display processing unit 130 may not display the measurement values of the corresponding measurement items in some of the first to P-th icons. The meaning of “each of the first to P-th icons” is the same in the following description.

Here, the first display region (E1) and the second display region (E2) indicate partial regions of the window of the detailed information or the display screen (A3) and are regions different from the first display region (D1) and the second display region (D2) of the display screen (A2 in FIG. 8 and the like) of the history information.

Here, the first to P-th measurement values may include at least one of the measurement values of a pace, a pitch, a stride, altitude information, a heart rate, and consumed calories in a workout.

The pace is information equivalent to a movement speed or a reciprocal of the movement speed. Here, a unit of a distance is set to km, a unit of a time is set to a minute and a second and various modifications can be made. Modifications of units of other measurement values can be made.

The pitch is information equivalent to the number of steps per unit time and a reciprocal of the number of steps. In swimming, the number of times of arm stroke (a stroke frequency) maybe used as an alternative of the number of steps. In the bike, information regarding cadence acquired from a bicycle computer may be used. The pitch is information obtained in accordance with the number of motions and a time for movement in a broad sense.

The stride is information indicating a movement distance (step size) per step. In swimming, a distance in which the user goes by stroking his or her arm once may be used. In the bike, a movement distance per rotation of a crank may be used. The stride is information obtained in accordance with the number of times of actions and a movement distance for movement in a broad sense.

The altitude information is information indicating an altitude corresponding to the position of the user. For example, an altitude acquired by a GPS receiver may be used. An absolute value (elevation) of the altitude may be used directly. In view of a considerable change in characteristics of a workout in an uphill slope or a downhill slope, information indicating a relative altitude which is a difference between a lowest altitude and a highest altitude along a route of the workout may be used. Alternatively, for the accumulative altitude, an accumulative value (accumulative ascending altitude) of a change in the elevation in an upward direction or an accumulative value (accumulative descending altitude) of a change in the elevation in a downward direction may be set as a measurement value.

The heart rate maybe acquired employing a heart rate sensor of a wearable device worn on a chest part of the user using a holding unit such as a belt. Alternatively, a heart rate acquired employing a biosensor (pulse wave sensor) of a wearable device worn on a wrist illustrated in FIG. 2 or the like may be set as the heart rate. Except for an exception such as a disorder, a pulse rate is known to be substantially identical to a heart rate.

FIG. 11 illustrates an example in which measurement values of the pace, the pitch, the stride, the altitude, the heart rate, and the consumed calories are included as the first to P-th measurement values. Any of the measurement values may be omitted or another measurement value may be added.

FIG. 11 illustrates an example in which the first axis of the graph is the horizontal axis and the second axis is the vertical axis. Therefore, the display processing unit 130 can set the pace, the pitch, the stride, the altitude, the heart rate, and the consumed calories on the vertical axis of the graph. The display processing unit 130 displays six icons (E21 to E26) for setting measurement items (measurement values) displayed on the vertical axis of the graph in the second display region (E2). E21 denotes an icon for setting a pace on the second axis, E22 denotes an icon for setting a pitch, E23 denotes an icon for setting a stride, E24 denotes an icon for setting an altitude, E25 denotes an icon for setting a heart rate, and E26 denotes an icon for setting consumed calories.

In each icon of E21 to E26, a measurement item name (the pace or the like) is displayed and a specific measurement value (6′ 23″/km or the like) of each measurement item is displayed. When an operation (a click operation or a tap operation in an icon display region) is executed on each icon, the display processing unit 130 sets a measurement value of the measurement item corresponding to the selected icon on the second axis. More specifically, each icon may be used as a toggle switch that switches setting/non-setting (display/non-display) of a corresponding measurement value on the second axis through an operation.

In the example of FIG. 11, the pace and the heart rate are set on the second axis. In FIG. 11, to clarify the measurement values set on the second axis, the corresponding icons (E21 and E25) are displayed with different colors from the other icons. In FIG. 11, the vertical axis on the left side of the graph of the first display region (E1) represents the pace and the vertical axis on the right side represents the heart rate, and vice versa. The chronological pace and heart rate acquired as workout information are displayed as polygonal line graphs (E11 and E15).

In this way, when the measurement values of the workout information are displayed as the graphs, measurement values which are display targets can be set (selected) using the icons (buttons) and can be displayed to be fitted to numerical values in the icon display regions. In a scheme of the related art, since setting icons have only button purposes, the icon regions maybe useless from the viewpoint of presenting the measurement values (numerical information) to the user. According to the scheme of the embodiment, the display regions can be effectively utilized by causing the icons to have both a first function such as a setting (selection) button and a second function such as display of a measurement value. Thus, it is possible to efficiently dispose numerical information of the measurement value in the restricted display screen and information of the graph.

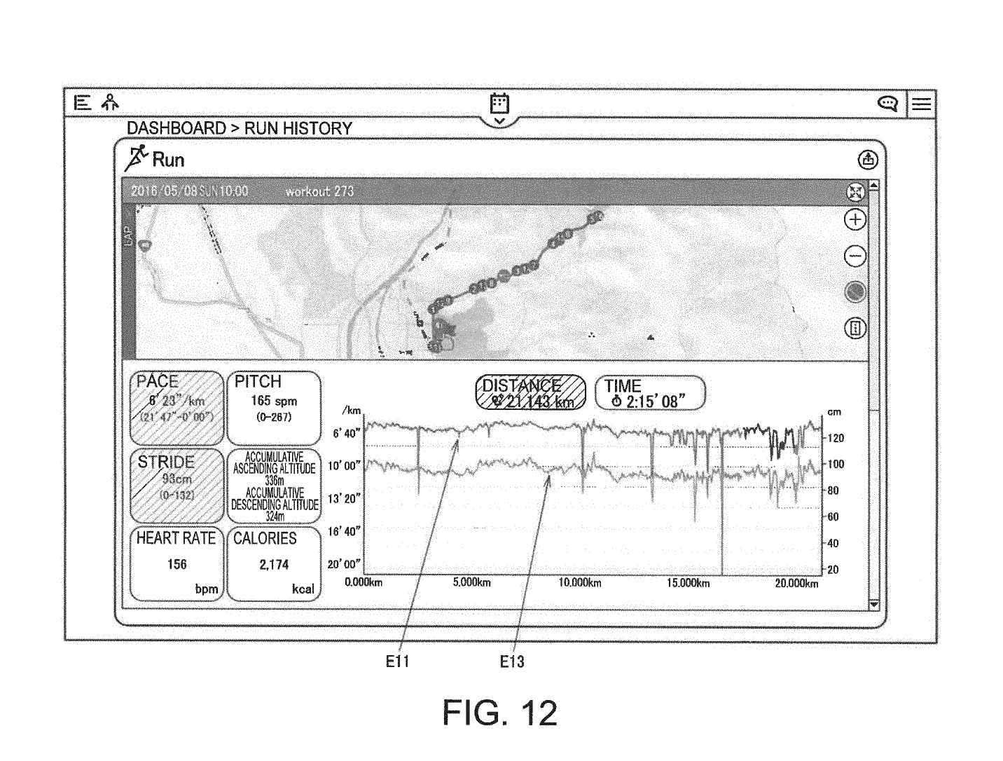

FIG. 12 illustrates a specific example of the display screen of the detailed information when an operation is executed on an icon which is not set. In the example of FIG. 12, a stride which is not displayed as a graph in FIG. 11 is set as a display target by executing an operation on the icon E23. Specifically, the vertical axis on the right side is switched to the stride and a chronological stride acquired as workout information is displayed as a line graph (E13).

FIGS. 11 and 12 illustrate examples in which the number of measurement items (measurement values) displayed as the graphs is restricted to 2. In this way, it is possible to prevent the display of the graph from being complicated and present the graph in the plain display form to the user. For example, when an operation of setting (displaying) a further different measurement value is executed in a state in which two measurement value have already been set (displayed), the display processing unit 130 may perform a process of performing non-setting(non-display) of one of the set two measurement values. In the example of FIG. 12, the heart rate is not set and the colors of the icons are changed to colors indicating the non-setting state. The measurement value in the non-setting (the non-display) maybe one of the two measurement values. For example, the measurement value in which the setting (display) starts at an earlier timing is considered not to be set.

In the embodiment, however, three or more measurement items (measurement values) are not hindered from being simultaneously displayed. Depending on a user operation, the number of measurement items which are the display targets is not hindered from becoming 1 or 0.

When a point or a range in the graph is designated, the display unit 150 (the display processing unit 130) may display according to the designation. When the point or the range is designated in the display of the graph, the display processing unit 130 displays the first to P-th measurement values in the designated point or range in regard to the first to P-th icons. Conversely, when the point or the range is not designated in the display of the graph, the display processing unit 130 displays statistical values obtained through a statistical process for the first to P-th measurement values during a workout period in regard to the first to P-th icons. Here, the workout period indicates a period from start to end of a workout in a narrow sense. When there is a period in which necessity for a target statistical process is high in a target workout, the period may be set as a workout period. Here, the workout period can be modified variously.

FIGS. 11 and 12 correspond to a state in which the point or the range is not designated. Therefore, the icons (E21 to E26) present information as an entire target workout. For example, an average value, a maximum value, and a minimum value of the pace are displayed in the icon E21. An average value, a maximum value, and a minimum value of the pitch are displayed in the icon E22. An average value, a maximum value, and a minimum value of the stride are displayed in the icon E23. An accumulative ascending altitude and an accumulative descending altitude in an entire workout are displayed in the icon E24. An average heart rate is displayed in the icon E25. A consumed calorie amount in an entire workout is displayed in the icon E26.

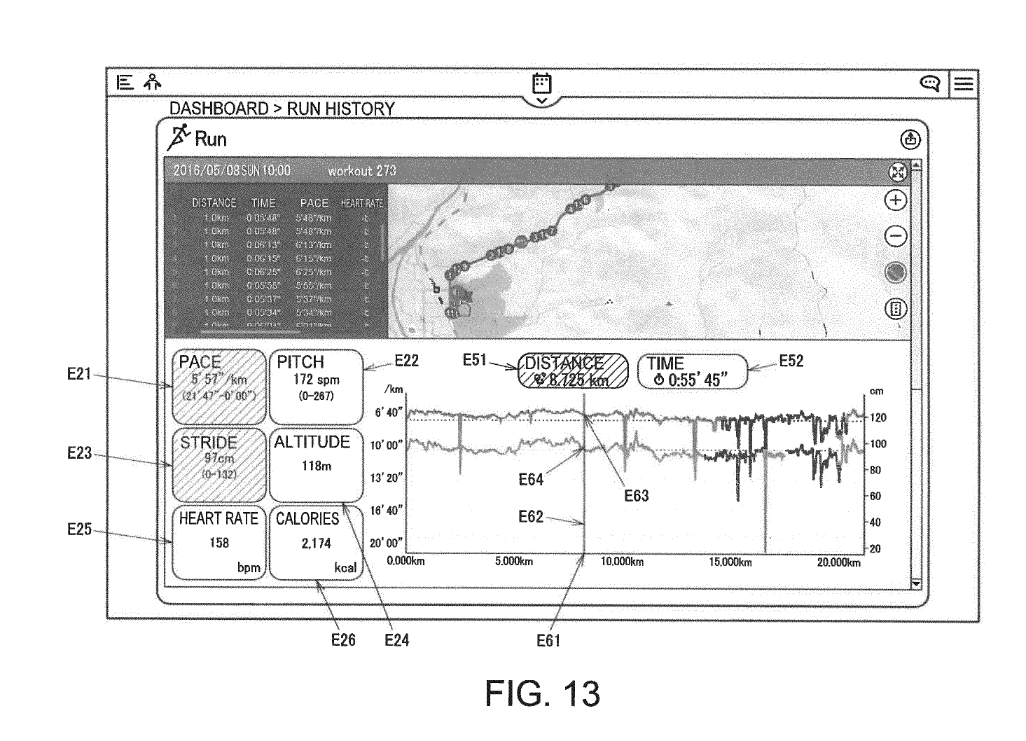

FIG. 13 illustrates a display screen example of detailed information in a state in which a point or a range is designated. In the example of FIG. 13, a given point (E61) in the horizontal axis direction in the graph of the first display region (E1) is designated. The display processing unit 130 displays a straight line (E62) vertical to the horizontal axis on the graph to clarify the position of the designated point in the horizontal axis direction. The designation in FIG. 13 can be realized, for example, through an operation moving a cursor to any position on the straight line of E62 in the first display region (E1) or an operation of tapping to the position.

In this case, the display processing unit 130 displays information regarding a measurement value corresponding to the point of E61 in the icon. For example, 5′ 57″/km which is a value (corresponding to E63) of the pace at the designated point is displayed in the icon E21. Similarly, 97 cm which is a value (corresponding to E64) of the stride at the designated point is displayed in the icon E23. About the measurement values (the pitch, the altitude, and the heart rate) which are not display targets in the graph, values at the designated point are displayed in the corresponding icons (E22, E24, and E25). Here, since the point is designated, an example in which an absolute value of the elevation is displayed in the icon (E24) indicating the altitude rather than a change in the altitude is illustrated. Here, since an example in which one value is obtained in regard to the consumed calories in an entire workout is illustrated, the display of E26 in FIG. 13 is the same as that in FIG. 11.

FIG. 13 illustrates an example in which the point is designated, but a range maybe designated. The designation of the range can be realized, for example, through a drag operation or a pinch-in operation. In this case, each icon may display a measurement value in the designated range. For example, the display processing unit 130 displays statistical values such as an average value, a maximum value, and a minimum value obtained based on measurement values in the designated range in the icons.

Although the description has been omitted above, the display processing unit 130 may display icons (E51 and E52) for selecting whether the first axis of the graph is set to either a distance or a time in a fifth display region (E5) of the display screen of the detailed information. In the example of FIG. 11, E51 is an icon corresponding to the distance and E52 is an icon corresponding to the time.

When the distance is set on the first axis, the left end of the graph indicates an exercise distance (a movement distance, 0 km) at a workout start position and the right end of the graph indicates an exercise distance (a total movement distance) at an workout end position. In the example of FIG. 11, the exercise distance in a workout is 21.143 km and the graph serves as a graph indicating how much a user moves in 21.143 km and what a measurement value is at the time of the movement.

When the time is set in the first axis, the left end of the graph indicates an exercise time (an elapsed time: 0:00′ 00″) at a workout start time point and the right end of the graph indicates an exercise time at a workout end time point.

In the example of FIG. 11, an exercise time of the workout is 2: 15′ 08″ and the graph indicates a change in a measurement value over time.

In the embodiment, numerical values are also displayed to be fitted in the icons E51 and E52. As in FIG. 11, when there is no designation on the graph and the exercise distance and the exercise time may be displayed in the entire workout, and the above-described 21.143 km and 2: 15′ 08″ are displayed.

When a point or a range is designated as in FIG. 13, information in the designated point or range maybe displayed. In the example of FIG. 13, the display processing unit 130 displays the fact that the point indicated in E61 corresponds to 8.725 km or 0: 55′ 45″ in the icon (E51 or E52). When a range is designated, the display processing unit 130 may display values at two end points in the range in the icons, for example.

### 5.2 Adjustment Object

The display unit 150 (the display processing unit 130) may receive a user operation and display adjustment objects (a controller and a component) for changing a range of the graph displayed in the first display region (E1) side by side with the graph in the first display region (E1). Here, the range is a display range on the first axis (the horizontal axis) in a narrow sense. Specifically, the range is a distance range or a time range which is a graph display target.

FIGS. 22 to 24 are diagrams illustrating specific examples of adjustment objects and a change in graph display based on operations on the adjustment objects. As indicated in E7 of FIG. 22, the adjustment object may be a bar-shaped object in which the horizontal axis direction of the graph is a longitudinal direction. The adjustment object (E7) includes a first object (E74) with a size corresponding to the width of the graph on the horizontal axis and a second object which is displayed in a part or the entirety of E74 and in which end portions (handles E71 and E72) and a middle portion (E73) are displayed to be recognizable. The size of E74 (the entire bar and a displayable range of the second object) is preferably identical to the width of the graph on the horizontal axis in consideration of comprehensibility of an interface, but the invention is not limited thereto.

The display processing unit 130 changes the display of the graph according to the position of the second object (a range of E71 to E73) in the first object (the displayable range: E74). Specifically, the display processing unit 130 displays data in the range indicated by E71 to E73 as a graph when the left endpoint of the entire bar (E74) on the horizontal axis is a starting point of a workout and the right end point of the workout is an ending point. More specifically, a point (a distance or a time) indicated as the left end point (the left handle: E71) of the second object is a starting point of the graph display and a point indicated as the right end point (the right handle: E72) is an ending point of the graph display.

As illustrated in FIG. 22, the second object corresponding to E71 to E73 is displayed in the entire E74 before an operation is received. FIG. 22 illustrates an example in which the distance is displayed on the horizontal axis. Therefore, in this case, a distance (0.000 km) corresponding to start of the workout is a starting point of the graph display and a distance (21.143 km) corresponding to end of the workout is an ending point of the graph display. In other words, a state in which no operation is executed on the adjustment object (in particular, the second object) is a state in which the range in the graph is not designated.

The adjustment object (E7: in particular, the second object) according to the embodiment can receive two kinds of operations by the user. When a first operation by the user on the adjustment object (E7) is received, the display processing unit 130 changes the length from the starting point to the ending point of the range by changing at least one of the starting point and the ending point of the range. Here, the first operation is, for example, an operation of moving the left end portion (E7 1) of the second object or an operation of moving the right end portion (E72).

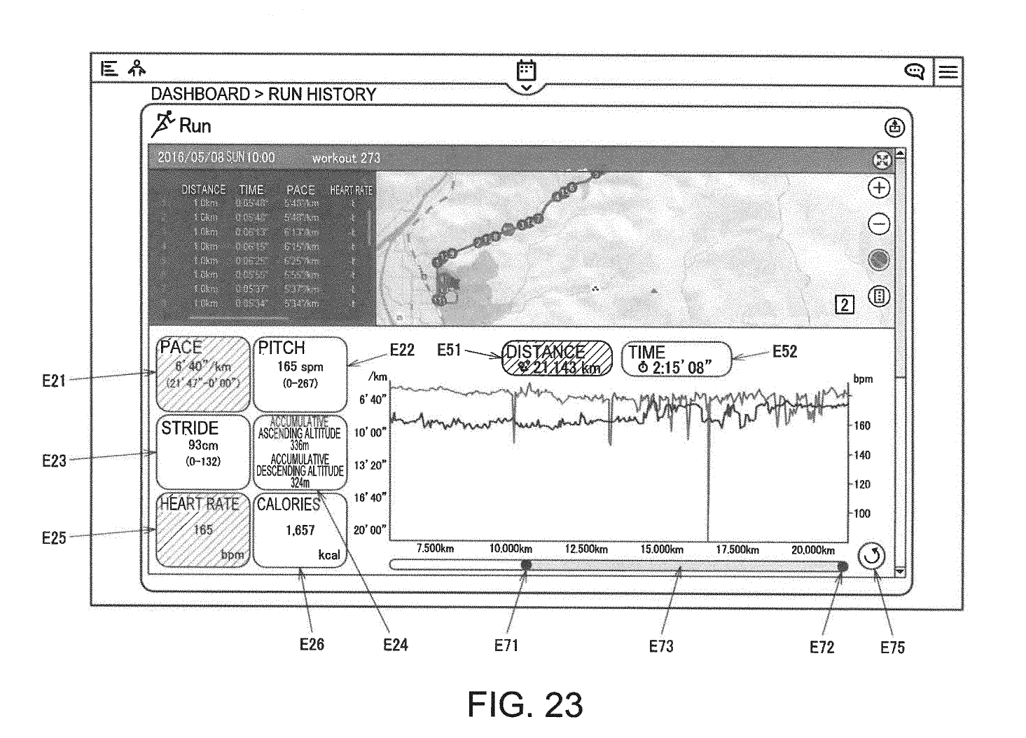

FIG. 23 illustrates an example of the display screen of the detailed information after the first operation of moving the left end portion (E71) of the second object to the right is received. As described above, the left end of E74 corresponds to 0.000 km and the right end corresponds to 21.143 km. In the example of FIG. 23, the left end portion (E71) of the second object is moved in the right direction from the left end of E74 and indicates a distance corresponding to 6,000 km. On the other hand, a state in which the right end portion (E72) of the second object indicates a distance corresponding to 21.143 km is maintained and E73 is displayed in a region between E71 and E72. When the operation illustrated in FIG. 23 is executed, the display processing unit 130 determines that a range of 6.000 km to 21.143 km is designated in the graph and sets the range to a display range (distance range) of the graph. Specifically, a part of the graph illustrated in FIG. 22 is expanded in the horizontal direction to be displayed. When the state of FIG. 22 (the length of the display range=21.143 km) is set as a reference, the length of the display range in FIG. 23 is 15.143 km. Therefore, an expansion ratio of the graph in the horizontal direction is 21.143/15.143≈1.4 times (about 140%).

In this way, the display range of the graph (the expansion ratio in the horizontal direction at the time of comparison with the state of FIG. 22) can be flexibly changed.

When the range is designated through a drag operation or a pinch-in operation of a mouse, there is a concern of minute adjustment being not easy after the range is designated. For example, it is necessary to designate the range again from the beginning after the range is returned to an undesignated state with a reset button. From this viewpoint, the designation of the range can be minutely and easily adjusted using the adjustment object (E7).

With the change in the display range (the change in the expansion ratio), each numerical value displayed in the second display region (E2) is changed to a value in the designated range. Numerical values displayed on the first axis (the horizontal axis) of the graph are changed in accordance with the display range. In the example of FIG. 23, the numerical values are changed at an interval of 2.500 km in accordance with the change in the expansion ratio. In the example of FIG. 23, in the display in the icons (E51 and E52), the values in the entire workout are maintained. However, as described above, the values at two end points of the display range may be displayed. The display in the six icons (E21 to E26) for setting measurement items (measurement values) and the display in the icons (E51 and E52) for selecting the first axis of the graph as one of the distance and the time can be modified variously. For example, even when the display range is changed (the expansion ratio is exchanged), a modification example can also be realized in which each numerical value displayed in the second display region (E2) is not changed (each value in the entire workout illustrated in FIG. 22 is maintained).

Here, the example in which the left end portion (E71) of the second object is moved to the right has been described. However, an operation of moving the right end portion (E72) to the left may be set as the first operation. The graph display may be contracted through an operation of moving the left end portion (E71) to the left after the expansion operation or an operation of moving the right end portion (E72) to the right, and this operation is included in the first operation. In FIG. 23, the example in which the right end portion (E72) is not moved when the operation is executed on the left end portion (E71) of the second object has been described, but the invention is not limited thereto. For example, when the left end portion (E71) is moved in the right direction by a predetermined amount, an operation interface in which the right end portion (E72) is moved in the left direction by the predetermined amount in association with the movement of the left end portion may be used.

The above-described first operation is an operation effective in designation of the expansion ratio (a length or a distance range from the starting point to the ending point). For example, when the user determines that visibility is bad due to considerable detail of the graph in which the entire workout (in the foregoing example, 0.000 km to 21.143 km) is a target, the user narrows the display range and expands the graph, so that the visibility of the graph can be improved. When the graph is considerably expanded, a contraction operation is also easy.

Here, in the first operation, a change in the expansion ratio is necessarily accompanied when the starting point or the ending point of the display range is changed. For example, 6.000 km is the starting point in FIG. 23. However, it is assumed that the user considers to desire to browse the graph from 3.000 km closer to the start. In this case, when the left end portion (E71) of the second object is moved to the left through the first operation, a display target is 3.000 km to 21.143 km and the expansion ratio is lower than that in the state of FIG. 23.

Accordingly, when the second operation of the user on the adjustment object is received, the display processing unit 130 maintains the length from the starting point to the ending point of the range and changes the starting point and the ending point. Here, the second operation is, for example, an operation of moving a middle portion (an adjustment bar: E73) of the second object to the right or left.

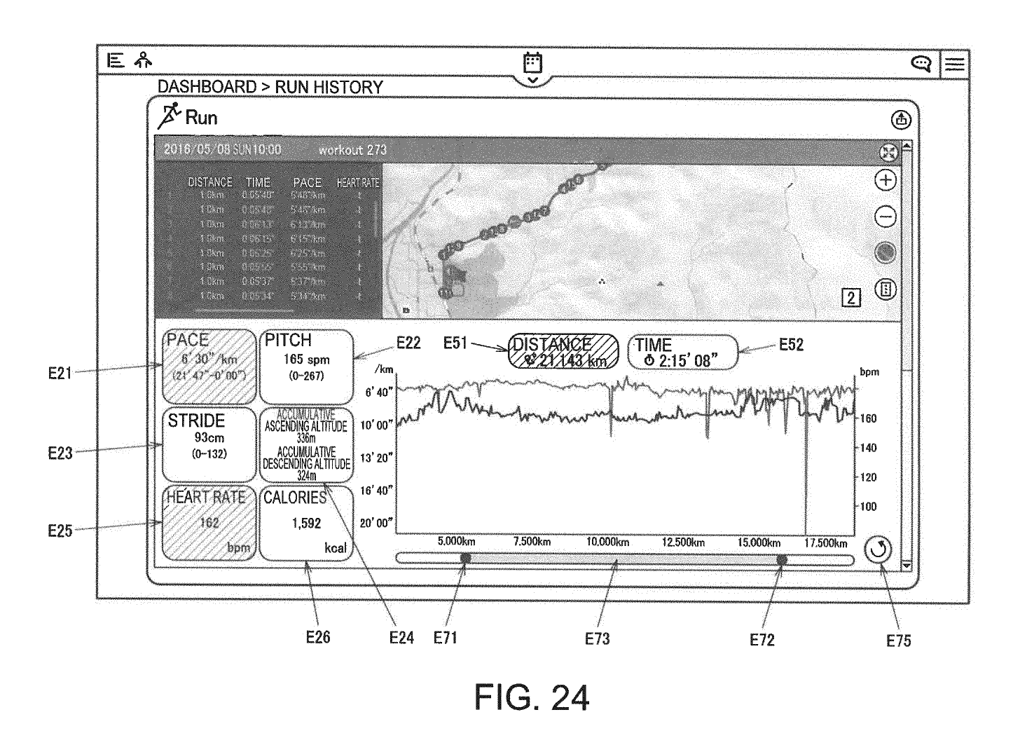

FIG. 24 illustrates an example of the display screen of the detailed information after the second operation of moving the middle portion (E73) of the second object to the left from the state of FIG. 23 is received. As understood from the comparison between FIGS. 23 and 24, when the operation is executed on the middle portion (E73), the size of E73 is maintained and the left end portion (E71) and the right end portion (E72) are moved in the same direction by the same amount. In this way, it is possible to flexibly change the position (the starting point and the ending point) of the display range while maintaining the expansion ratio of the graph. Since E71 corresponds to 3.000 km and E72 corresponds to 18.143 km in the example of FIG. 24, the expansion ratio in the state of FIG. 23 is maintained.

The adjustment of the distance range has been described above. The same adjustment is also possible even when the horizontal axis is time. The adjustment object may include a reset button (E75), as illustrated in FIGS. 23 and 24. The reset button (E75) is a button object displayed when the expansion ratio increases from an equal magnification (the magnification 100%), that is, when the display range is narrower than the entire workout, and receives a pressing operation. When an operation of pressing the reset button is received, the display processing unit 130 performs a process of returning the expansion ratio to the equal magnification state (FIG. 22).

### 5.3 Map Information and Lap Information

The display processing unit 130 displays map information related to a workout in the third display region (E3). In this way, it is possible to present where the user executes a workout in an intuitively comprehensible display mode. Here, the map information may be information in which movement route information is superimposed on a map, as illustrated in FIG. 11.

The display unit 150 (the display processing unit 130) may also display lap information of a workout in a fourth display region (E4). A lap is information indicating time (lap time) necessary for movement of a predetermined section. Here, the predetermined section may be, for example, a section of a predetermined distance (1 km, 1 mile, or the like). Alternatively, the predetermined section may be a section in which the user can arbitrarily set a starting point and an ending point. For example, when the user operates the wearable device 200 at the time of passing a check point, the endpoints of the predetermined period may be set. Here, the lap information is information that includes at least the above-described lap time. In the example of FIG. 11, the lap information is information such as a distance of the predetermined section, a lap time, an average pace in the predetermined section, or an average heart rate in the predetermined section.

Here, the lap information is information in the predetermined section, as described above. Therefore, information in which the predetermined section means where to where is important. For example, there is, of course, a difference between a lap time in a flat section and a lap time in an uphill section. In this case, it is not preferable to simply compare the lap time. That is, the lap information is not displayed solely, but is preferably displayed in accordance with information regarding a target section.

Accordingly, in the embodiment, the third display region (E3) and the fourth display region (E4) are displayed on the display screen in a positional relation in which the regions are within one screen. The map information includes information indicating a movement route of the user. Therefore, by displaying the lap information and the map information to be fitted, it is possible to realize display in which the user can intuitively understand the section corresponding to each lap.

At this time, a number used in association with the lap information may be displayed on the route other than displaying only the route. In the example of FIG. 11, numbers are attached to the lap information of sections displayed in a list form in the vertical direction and corresponding numbers are also displayed in the map information. In this way, it is possible to comprehensibly present the correspondence between the lap information (a lap time or the like) and the section in which the lap information is acquired. The correspondence is not limited to the scheme in which numbers are used, but can be realized in various forms such as alphabet letters.

For example, as illustrated in FIG. 11, the fourth display region may be disposed to overlap (superimpose) with the third display region. In this way, since the map information and the lap information are displayed inevitably at close positions, display which the user can easily see is possible. Alternatively, the fourth display region may be disposed to be adjacent to the third display region. In this case, when a boundary object indicating a boundary of a display region is disposed between the third and fourth display regions and the reception unit 110 receives an operation on the boundary object, the size of display of the third and fourth display regions or an occupation ratio in a display screen can be changed.

Here, even the map information alone is useful display which is easy to intuitively understand. Therefore, it is sufficiently considered that the user browses map information in a large display region. In this case, it is not preferable that the map information is blocked by the lap information when the fourth display region overlaps with the third display region. Even when the lap information is transmissively displayed, visibility of the map information in the overlapping region may deteriorate.

Accordingly, the display unit 150 (the display processing unit 130) may switch the fourth display region between non-display and overlap display with the third display region in response to a user operation received by the reception unit 110.

FIG. 12 is a diagram illustrating switch between display modes of the fourth display region. In FIG. 12, the fourth display region (lap information) is not displayed. In this way, appropriate display can be realized depending on a situation of the user who desires to browse the fit lap information and map information or desires to take notice of the map information.

An operation of realizing non-display of the lap information can be realized through, for example, a click operation, a tap operation, or a slide operation on a given region of the fourth display region. An operation of realizing overlap display of the fourth display region can be realized through, for example, a click operation, a tap operation, or a slide operation on a given region (in the example of FIG. 12, a strip region at the left end portion) of the third display region.

From the viewpoint of displaying the map information in a broad region, the third display region can also be expanded.

Specifically, when the reception unit 110 receives an expansion operation by the user, the display processing unit 130 expands and displays the third display region and moves the first and second display regions in a predetermined direction.

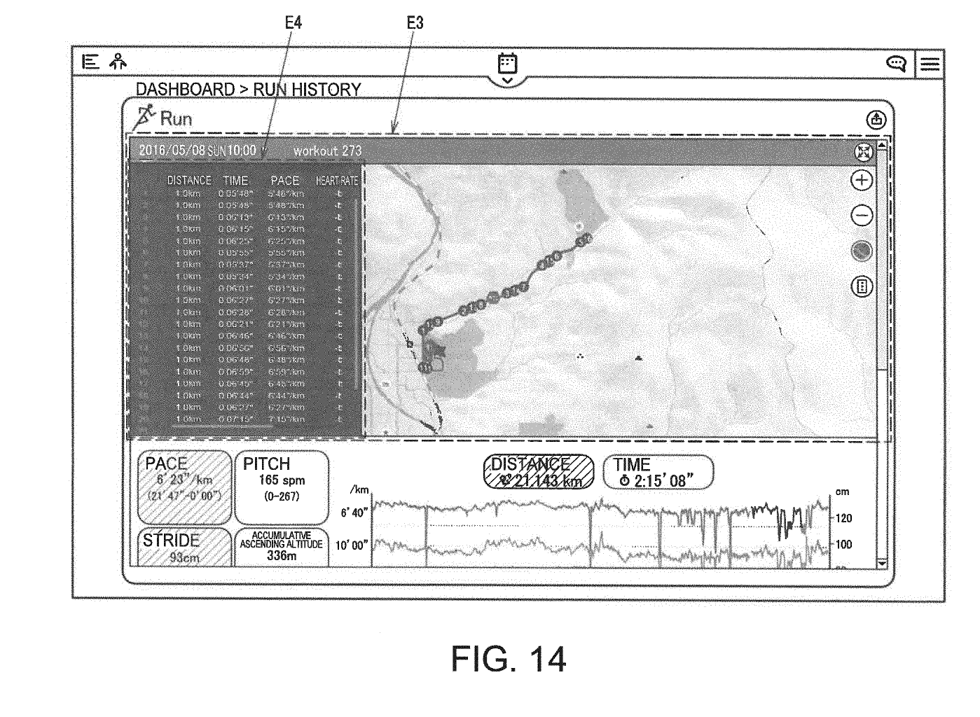

FIG. 14 illustrates a specific example of the display screen of the detailed information when the expansion operation is received. Various specific examples of the expansion operation are considered. For example, pressing (clicking or tapping) an expansion button denoted by E7 of FIG. 11 is used as the expansion operation. As understood from the comparison between FIGS. 11 and 14, the third display region is expanded (in the example of FIG. 14, the size is expanded in the vertical direction). Accordingly, the first and second display regions are moved in the downward direction. In this way, it is possible to improve the visibility of the map information and appropriately adjust a positional relation between the graph of measurement values and display of numerical values.

When the third display region is expanded and displayed, the display processing unit 130 may further increase at least one of the number of pieces of the lap information and an amount of information displayed in the fourth display region than when the third display region is not expanded to be displayed.

In the example of FIG. 14, the fourth display region is also expanded in the vertical direction to be fit to the expansion of the third display region. Accordingly, the number of pieces of lap information displayed at a time is increased from 8 to 20. FIG. 14 illustrates an example in which the number of pieces of lap information is increased, but the amount of information may be increased. For example, the display processing unit 130 may display information different from the distance, the lap time, the average pace, and the average heart rate as lap information. In this way, it is possible to expand the map information and enrich the lap information presented in the third display region.

As illustrated in FIG. 11 and the like, the first display region (E1), the second display region (E2), the third display region (E3), and the fourth display region (E4) are preferably arranged in one display screen (window). The graph of the measurement values, the numerical values (the icons), the map information, and the lap information are useful as the information indicating a workout of each user. However, in the scheme of the related art, it is difficult to browse all of the information unless scrolling or screen transition is executed and there is a problem of convenience. From this viewpoint, in the embodiment, by using the display of the numerical values in the setting icons or the overlap of the lap information with the map information, it is possible to efficiently dispose each piece of information and arrange the four display regions on one display screen. That is, the user is caused not to execute a complicated screen transition operation and display for browsing various kinds of information can be realized.

In the scheme according to the embodiment, display in the first and second display regions is not requisite. That is, in the scheme according to the embodiment, the reception unit 110 that receives an operation by the user, the storage unit 120 that stores workout information based on a workout executed by the user, and the display unit 150 that displays the workout information are included. The display unit 150 (the display processing unit 130) can be applied to the workout information display system 100 that overlaps and displays the map information and the lap information associated with the workout.

Even in this case, as illustrated in FIG. 12, the display processing unit 130 may switch non-display of the lap information and overlapping display of the lap information with the map information in response to a user operation received by the reception unit 110.

### 6. Similar Workout Information

### 6.1 Example of Screen on Which Similar Workout Information is Displayed

By using the board screen (A1), the display screen (A2) of the history information, and the display screen (A3) of the detailed information described above, it is possible to appropriately display the workout information regarding a workout executed by the user. The advantages of each screen have been described above.

Here, the user who executes a workout is considered to be strongly interested in how much the performance of the user is improved by accumulation of workouts. In this case, it is not possible to ascertain a change in the performance by browsing the detailed information of a given workout of one time. It is not easy to measure a muscle strength or a cardiorespiratory ability and it is difficult to use the muscle strength and the cardiorespiratory ability as index values of the performance daily.

On the other hand, a scheme of presenting a change in the performance of the user by comparing a plurality of pieces of workout information is considered. For example, when a time necessary to run a given course is shortened, it is possible to determine that the performance of the user is improved.

At this time, it is necessary for the plurality of comparison target workouts (an example in which two workouts will be described below) to have similar characteristics. For example, for two workouts in which exercise distances are different, exercise times are, of course, different and it is difficult to determine a change in the performance despite comparison. Even when the exercise distances are the same, exercise times are, of course, different between a flat course and an uphill course and the comparison meaning is weak. That is, it is necessary for the plurality of pieces of comparison target workout information to be mutually similar.

A scheme in which the system automatically compares the plurality of pieces of similar workout information and display a change in the performance can also be considered. However, an amount of information regarding whether the performance is improved is small. For example, it is preferable to consider to separate the improvement of the performance into an improvement in a speed in a short distance of about several km and a gain of stamina for a long distance of about several tens of km. Alternatively, separating the improvement of the performance into an improvement in a speed in a flat route and a reduction in a time loss in ups and downs can also be considered. That is, since various forms of the improvement in the performance are considered, it is insufficient to compare workout information and display only a comparison result (for example, only a time difference) and it is preferable to present information detailed to some extent in regard to each piece of workout information.

An intention of the user is, of course, reflected in selection of the comparison target workout information. For example, a workout executed after elaborate adjustment just as an actual race is considered to have higher importance than a workout which is executed daily. In this example, the user is assumed to desire to compare workouts with high importance. When there are courses which the user likes or does not like, a request for setting a workout in the course which the user like as a comparison target can also be considered. In consideration of such a viewpoint, it is preferable to leave space for selection by the user rather than the system automatically selects comparison workout information from the viewpoint of non-similarity.

In view of the above description, when the reception unit 110 receives an operation of selecting selection workout information from a plurality of pieces of workout information in the workout information display system 100 according to the embodiment, the processing unit 140 specifies similar workout information satisfying a given similarity condition between the selection workout information from the workout information other than the selection workout information and the display unit 150 displays the selection workout information and the similar workout information. Specifically, the display processing unit 130 may perform a process of displaying the selection workout information and the similar workout information on the display unit 150.

Here, the processing unit 140 may perform a process of requesting (extracting) similar workout information in real time or may read the pre-requested similar workout information from the storage unit 120. Here, the real time indicates that the process of requesting (extracting) the similar workout information starts when the reception unit 110 receives an operation serving as a trigger of the display of the similar workout information. The details of the process of requesting the similar workout information will be described below. The process of requesting the similar workout information may be performed by another device (for example, the server system 500 when the workout information display system 100 is included in the mobile terminal device 300). In this case, the similar workout information requested by the server system 500 may be downloaded in advance to the storage unit 120 and the processing unit 140 may read the similar workout information. Alternatively, the processing unit 140 may perform the process of requesting the similar workout information in real time from the server system 500. The process of specifying the similar workout information according to the embodiment is included in such a process.

In this way, since the similar workout information (in a narrow sense, workout information regarding a workout in a similar course, as will be described below) is automatically extracted, a labor of the user can be reduced. Since only a change in the performance is not displayed and a comparison screen of the selection workout information and the similar workout information are displayed, it is possible to allow the user to refer to detailed data and evaluate the performance based on the reference. Further, since the user is allowed to select at least the selection workout information, the workout information in which the user is strongly interested can be included in a comparison target.

When the plurality of pieces of similar workout information are specified, the display unit 150 may display the plurality of pieces of specified similar workout information (in a narrow sense, list display).

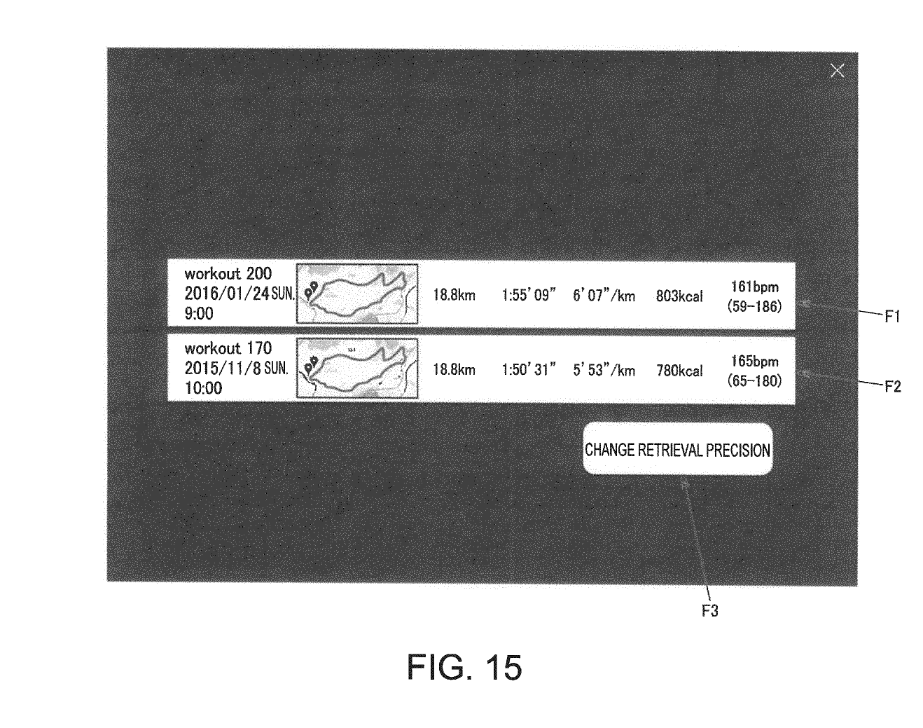

FIG. 15 illustrates an example of a screen on which a list of the similar workout information is displayed. Here, two pieces of similar workout information are specified and a list of the two pieces of similar workout information (F1 and F2) is displayed. A display mode of each piece of similar workout information may be the same as that of, for example, the first display region (D1 in FIG. 8 and more specifically, D32, D33, and the like) on the display screen of the history information. The screen of FIG. 15 is assumed to be a pop-up screen displayed to overlap with the display screen of the detailed information in FIG. 10, but the invention is not limited thereto. For example, the display processing unit 130 may end the screen display of FIG. 10 and perform screen transition to display the screen of FIG. 15 or maybe configured to display the similar workout information extracted in the first display region of the screen display of FIG. 10.

The similar workout information may include map information. For example, as illustrated in FIG. 15, since the map information is included in the list of the displayed similar workout information and content of a workout corresponding to the similar workout information can be intuitively estimated, appropriate workout information can be compared.

When the reception unit 110 receives an operation of selecting one of the list of the displayed similar workout information, the display processing unit 130 performs a process of displaying a comparison screen of the selected similar workout information and the selection workout information.

In this way, the user can also be committed to selecting the similar workout information. That is, the system side performs complicated determination of similarity or non-similarity and candidates (similar workout information) for the comparison target workout information are narrowed down. However, final selection is executed by the user. In this way, since user's determination can be used for both the selection of the selection workout information and the selection of the similar workout information, the workout information can be compared as intended by the user.

FIG. 16 illustrates an example of a comparison screen on which selection workout information is compared to similar workout information. FIG. 16 is a diagram illustrating a list of information displayed on the comparison screen. It is not necessary to display simultaneously all of the information in FIG. 16. For example, the display processing unit 130 may perform scroll display of images illustrated in FIG. 16.

As illustrated in FIG. 16, the comparison screen of the selection workout information and the similar workout information includes at least comparison display for routes and comparison display for measurement values. The measurement value comparison display includes comparison display for graphs and comparison display for numerical values. Here, the measurement values may be information including at least one of a pace, a pitch, a stride, an altitude, a heart rate, and consumed calories of the workout as in the display screen of the detailed information in FIG. 11. In FIG. 16, the yard-pound system may be used in display of the measurement values, but a metric system may be used as in FIG. 11. The display processing unit 130 may switch a display mode of the measurement values (a unit system, a font, a language, and the like) in response to a user operation.

G1 of FIG. 16 denotes the comparison display for routes. Specifically, both route information of the selection workout information and route information of the similar workout information are displayed to overlap with the map information. In a modification example to be described below, a case in which places where two workouts are executed are separated can also be considered. In this case, two pieces of route information may not overlap with one piece of map information, but two pieces of map information may be displayed side by side.

G2 and G3 denote the comparison display for the measurement values. G2 corresponds to the comparison display of graphs and G3 corresponds to the comparison display of numerical values. G3 is the same icon as E21 to E26 in FIG. 11. That is, each icon of G3 is a button for selecting a measurement item (measurement value) displayed in a graph of G2 and is a region in which a measurement value (numerical value) is displayed. Here, in each of the selection workout information and the similar workout information, an example in which the same six items as those of FIG. 11 can be selected is illustrated. Therefore, twelve icons are displayed in G3. In G2, graph display in which three measurement values of a stride, an altitude, and a heart rate are compared is realized, but the number of measurement values displayed as in FIG. 11 maybe limited to two. As indicated in G2, it is easy to compare the measurement values when the measurement values of the selection workout information and the measurement values of the similar workout information are displayed in one graph. Here, a modification can also be realized in which two graphs are displayed side by side.

G4 is display of the same lap information as E4 in FIG. 11. In the example of FIG. 11, a lap is measured in both the selection workout information and the similar workout information by using a section of 1.2 miles as a unit, and thus comprehensible comparison is possible. Here, a predetermined section which is a measurement target of the lap information can be set arbitrarily and the predetermined sections of the selection workout information and the similar workout information may not identical to each other. As in FIG. 11, the lap information may be displayed to overlap with the map information. Here, note that there is a concern of visibility of the map information deteriorating when two pieces of lap information overlap with each other.

G5 denotes display of a maximum oxygen intake VO2max. VO2max indicates a maximum amount of oxygen inhaled in a body during an exercise and can be estimated from body motion information or biometric information. Since VO2max is an index value for ascertaining an exercise intensity of the user, VO2max is suitable as information at the time of comparing two workouts intuitively. Expected times may be displayed based on a value of VO2max when the user runs by each distance (5 miles, 10 miles, a half marathon, a marathon, and the like).

G6 denotes display of a heart rate zone. The heart rate zone is information indicating that a heart rate is divided into several numerical ranges (zones) and indicating the length of a stay time in each zone. The heart rate zone is also suitable as an index value for ascertaining the exercise intensity of the user. As in the example of FIG. 16, by displaying a stay time of each zone in a graph, display for facilitating visual understanding is also possible. Although not illustrated in FIG. 11, VO2max or the heart rate zone is not hindered from being displayed on the display screen of the detailed information of one piece of workout information.

By using the comparison screen in FIG. 16, it is possible to appropriately compare the selection workout information to the similar workout information. In particular, in the example of FIG. 16, the same items as those of the detailed information in FIG. 11 are display targets in each piece of workout information. Therefore, detailed comparison of two pieces of workout information can be performed and the user can be allowed to appropriately determine a change in the performance.

Various triggers for starting the display of FIG. 15 or 16 are considered. For example, the display unit 150 may display an icon regarding the similar workout information on a screen on which the detailed information of the selection workout information is displayed. When the reception unit 110 receives an operation on the icon, the display unit 150 may display the similar workout information (display the list of the similar workout information).

In the example of FIG. 11, an icon E8 is displayed in a part of the third display region (E3) in which the map information is displayed. As illustrated in FIG. 11, when a similar workout is detected, the number of pieces of similar workout information may be displayed in the icon. When no similar workout is detected, “0” may be displayed and the icon E8 may not be displayed. The user may be notified that there is a similar workout using a sentence “There is similar workout” rather than the icon. In this way, before the display of the specific list screen (FIG. 15), it is possible to present presence of the similar workout information and the number of pieces of similar workout information to the user.

### 6.2 Process of Retrieving Similar Workout Information

Next, a specific example of a process of retrieving (selecting or extracting) the similar workout information will be described. Hereinafter, the process is performed by the processing unit 140. However, a modification can be made, as described above. In the embodiment, positional information of the user is acquired when the user executes a workout. In particular, when the wearable device 200 includes a GPS receiver, an absolute position indicated using latitude and longitude can be acquired. Therefore, the processing unit 140 sets the positional information as a feature amount and determines similarity between the workout information.

For example, the similarity condition herein includes kinds of workouts. Even when the user moves the same course, comparison between workout information of a run and workout information of a bike may not be said to be meaningful. Accordingly, when the selection workout information is information regarding a run (in a broad sense, a first kind of run), the similar workout information may be selected from workout information of the run (first kind of run).

In a simplest way, comparing two workouts executed in the same course as a target can be said to be effective. This is because an influence of a course on measurement values is small in a workout of the same course. In other words, when a change in the measurement values occurs, the change can be estimated to occur due to a difference in the performance of the user.

Accordingly, the similarity condition is assumed to be a condition regarding route information of the workout. The similarity condition may include a starting position and an ending position of the workout. For example, the processing unit 140 compares the start positions of two pieces of workout information and sets a condition that an error is within a radius of 200 m as the similarity condition. Similarly, for the ending position, a condition that an error is within a radius of 200 m is set as the similarity condition. Of course, the threshold herein is not limited to 200 m and various values can be set.

The similarity condition may include an exercise distance of a workout. When the exercise distances are considerably different, it is expected that a load on the user is considerably different in two workouts. Therefore, comparison meaning is weak. Accordingly, the processing unit 140 sets a condition that an error of the exercise distances of two workouts is within ±10% as the similarity condition. The threshold can also be modified.

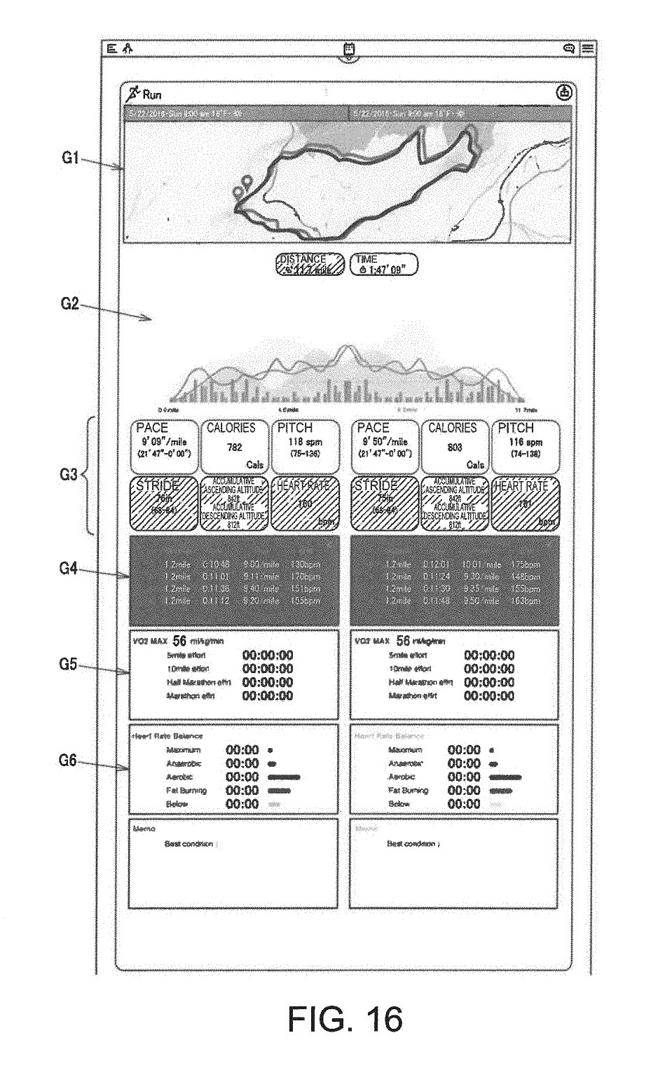

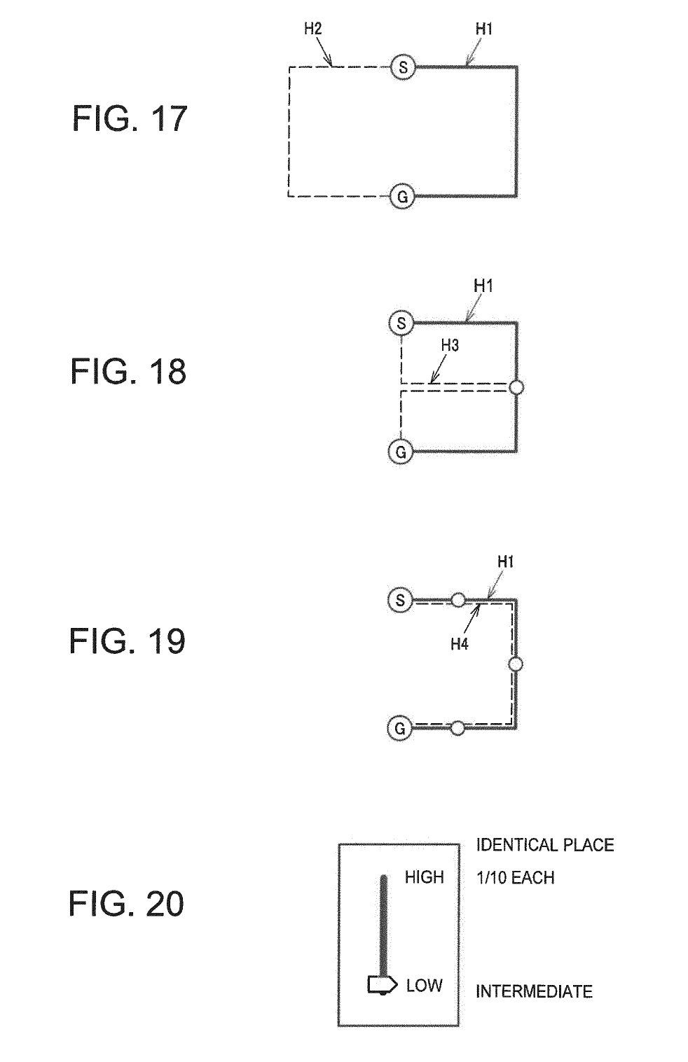

FIG. 17 illustrates specific examples of a route (H1) of the selection workout information and a route (H2) of the similar workout information determined to be similar in accordance with the foregoing similarity condition. In FIG. 17 and FIGS. 18 and 19 to be described below, S denotes a starting position of a workout and G denotes an ending position. As illustrated in FIG. 17, there is a concern that workout information in which a route is considerably different is determined to be similar only with a starting position, an ending position, and a distance.

Accordingly, the similar condition preferably includes an intermediate point between the starting position and the ending position. For example, the processing unit 140 compares two intermediate points of two pieces of workout information and sets a condition that an error is within a radius of 200 m as a similar condition. In this way, the route illustrated in H2 of FIG. 17 can be excluded since the route does not satisfy the similarity condition.

FIG. 18 illustrates specific examples of the route (H1) of the selection workout information and a route (H3) of the similar workout information when one point is set as an intermediate point. FIG. 19 illustrates specific examples of the route (H1) of the selection workout information and a route (H4) of the similar workout information when three points are set as intermediate points.

As understood from comparison between FIGS. 18 and 19, there is a concern of separation being considerable between the route (H1) of the selection workout information and the route (H3) of the similar workout information when the number of intermediate points is small. As the number of intermediate points is larger, there is a high possibility of the route (H1) of the selection workout information being near the route (H4) of the similar workout information. Accordingly, in consideration of precision of the similarity determination, it is preferable to increase the number of intermediate points.

However, even when the same similar condition is used, the number of pieces of similar workout information is changed due to characteristics of the selection workout information or a history of workouts executed in the past by the user. For example, first selection workout information may be determined to be similar to many pieces of workout information when the similar workout information is retrieved under a given similarity condition. In this case, since the number of pieces of similar workout information displayed in as a list on the screen of FIG. 15 is large, there is a concern of the selection of the user being complicated. On the other hand, second selection workout information is determined to be similar to only a small number of pieces of workout information when the similar workout information is retrieved under the same similarity condition. In an extreme case, it is considered that there is no similar workout information and appropriate comparison is difficult.

In consideration of the foregoing point, the reception unit 110 may receive operation information for changing the similarity condition. In this way, it is possible to flexibly set the similarity condition depending on a situation. For example, when there is a large number of pieces of similar workout information, the similarity condition may be strict. When there is a small number of pieces of similar workout information, the similarity condition may be loose.

As understood from the example, the user can generally estimate whether the current similarity condition is appropriate by browsing the list display screen of FIG. 15. Accordingly, the display processing unit 130 may be able to change the similarity condition from the screen (FIG. 15) on which the list of the similar workout information is displayed.

In the example of FIG. 15, a button “Change retrieval precision” is displayed in the lower portion of the list display. When the button is pressed, the screen transitions to a screen for changing the similarity condition.

FIG. 20 illustrates a specific example of a screen on which the similarity condition is input. In FIG. 20, a slider is displayed. When the slider is moved to one side (the upper side in the example of FIG. 20), the similarity condition becomes strict. When the slider is moved to the other side (the lower side), the similarity condition becomes loose.

At this time, the processing unit 140 may variably set the number of intermediate points in response to a user operation. As illustrated in FIGS. 17 to 19, the precision of the similarity determination (retrieval precision) is improved as the number of intermediate points is larger. Accordingly, the number of intermediate points can be said to be appropriate as a parameter for adjusting difficulty of the similarity condition. Even on the screen of FIG. 20, identical places of routes in the selection workout information and the similar workout information, that is, the number of intermediate points, increase as the slider is moved upwardly. The number of intermediate points decreases as the slider is moved downwardly. In the case of FIG. 20, the slider sets a ratio at which the intermediate points are provided. One intermediate point is set at an intermediate position of the route in the lowest portion. When the slider is in the highest portion, the route is divided into 10 points and the intermediate point is set at each divided point.

The foregoing process is an example of the similarity determination and a known matching algorithm can be broadly applied to the similarity determination of the route. Since a parameter for determining the retrieval precision is different depending on a matching algorithm, the processing unit 140 can change the similarity condition by changing various parameters.

### 6.3 Modification Examples

As described above, the route information (in particular, positional information) is used as the similarity condition. Therefore, a workout in a different place is determined not to be similar.

However, when the exercise distances are the same, a request for comparing workouts in which positions or routes are different and browsing the workouts is considered. For example, a user who is a marathon runner may desire a comparison between exercise information in Tokyo Marathon and workout information in Honolulu Marathon. In Tokyo Marathon and Honolulu Marathon, a flat distance of a course is long although ups and downs of about 30 m are seen several times. That is, the position and the route are considerably different, but it is meaningful to compare results of the two marathons. In particular, when a starting position, an ending position, and an intermediate point are set as similarity condition in a case of a workout in which a distance is long as in marathon, similar workout information is assumed to be acquirable only once in each race (for example, once per year). Therefore, the meaning of the foregoing comparison is considerable.

In this case, the processing unit 140 retrieves the similar workout information using an exercise distance of the workout as the similarity condition. The retrieval is particularly effective when the number of pieces of similar workout information is extremely small as in the example of marathon. Here, since necessity for including courses of which characteristics are considerably different as comparison targets is low, ups and downs (an altitude, in a narrow sense, an accumulative ascending altitude and an accumulative descending altitude, or obliquity (the degree of altitude change per unit distance)) may be added to the similarity condition depending on a case.

Alternatively, a pace may be added to the similarity condition in addition to the distance and the ups and downs. When the workouts in similar routes are compared, the magnitude of a value of the pace is information which is used to determine performance. Here, by incorporating the pace into the similar condition, courses of the same ups and downs at the same distance can be set as targets and data can be compared when a workout of the same pace is executed. In this case, information with a large comparison meaning on the comparison screen is a heart rate (an average value, an increase amount, or the degree of increase per unit time).

Two workouts in which the distance, ups and downs, and the pace are similar are considered to have the same load of the workouts on the user. In such a presupposition, when how internal information (biometric information) regarding the user responds is compared, an ability of an organism of the user, specifically, a change in a cardiorespiratory ability, can be estimated. When the cardiorespiratory ability is high, a change in a heart rate on the same load is suppressed to be small. That is, by comparing heart rate information under such a similarity condition, it is possible to determine whether the cardiorespiratory ability is improved or whether the user is in a slump. As the similarity condition, the similar workout information can also be extracted setting at least one of a distance, ups and downs, and a pace as the condition.

The board screen is configured such that the user can arbitrarily generate cards. For example, the user may designate arbitrary workout information as “favorite”, “my best”, or “benchmark” among the history information and display a card for displaying workout information corresponding to the designated workout information (designated workout information) as a benchmark card. When the workout information received by the reception unit is a similar workout to the designated workout information, the comparison result maybe displayed with a card. For example, when the reception unit receives most recent workout information satisfying the similar condition of the designated workout information and the designated workout information, a comparison result of the designated workout and the most recent workout can be displayed with a card corresponding to the most recent workout or a card corresponding to the designated workout information.

### 7. Calendar Screen

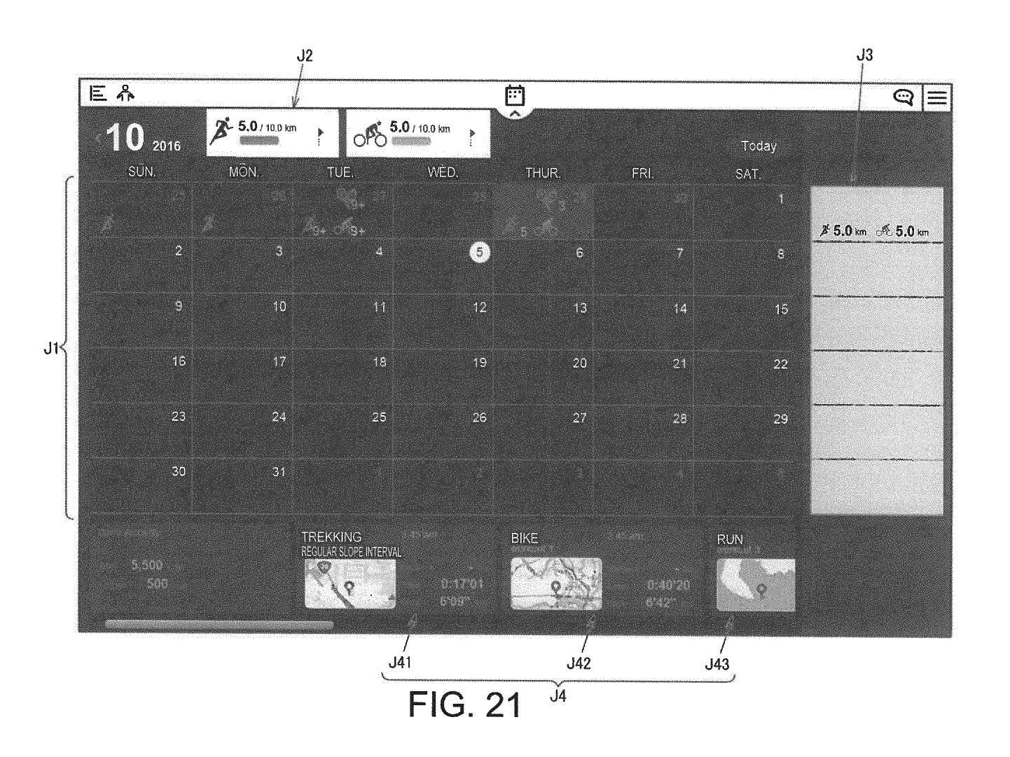

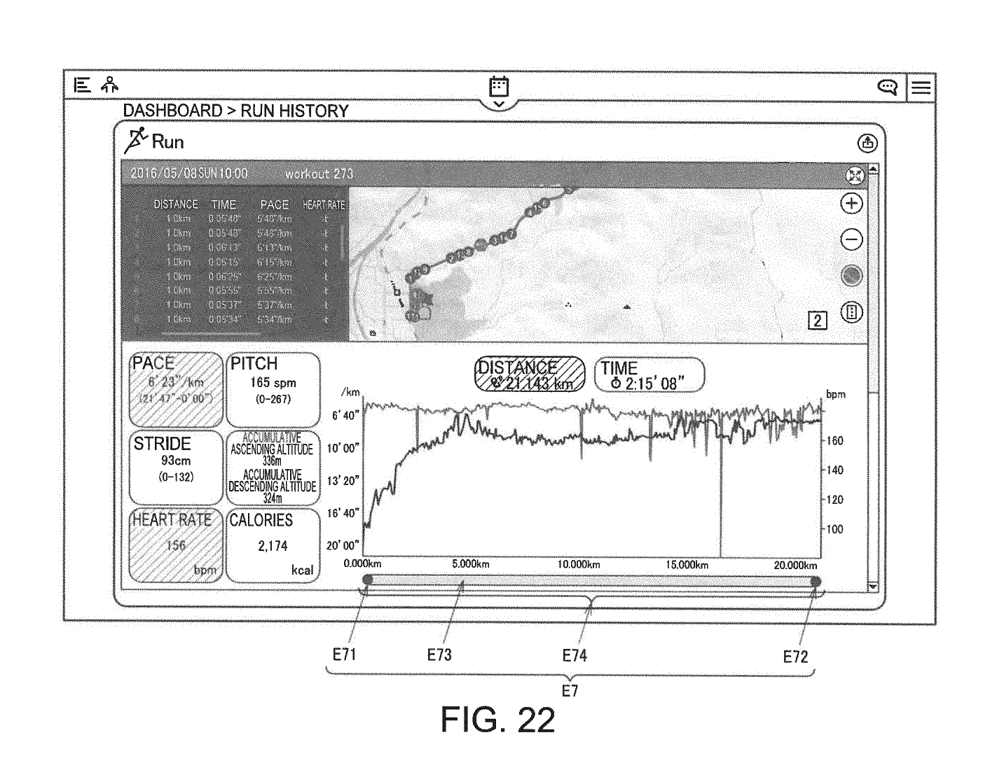

FIG. 21 illustrates a specific example of a calendar screen illustrated in A6 of FIG. 5. In the example of FIG. 21, a calendar of one month is displayed (J1) and an icon corresponding to a kind of workout is displayed on a day on which the workout is executed. Statistical information (for example, a sum value of exercise distances) in which one displayed month is set as a target is displayed (J2) for each kind on the upper side of the calendar. Statistical information in which one corresponding week is set as a target is displayed (J3) on the right side of the calendar.

When an operation of designating one day of the calendar is executed, information regarding a workout executed on the designated day is displayed in the lower portion of the calendar (J4). In the example of FIG. 21, workout information regarding a trekking (J41), a bike (J42), and a run (J43) is displayed. As items displayed in J4, for example, a date, map information, an exercise distance, and an exercise time may be used as in the history information (D32 and the like) of FIG. 8.

By using the calendar, it is possible to search for the workout information from a date on which a workout is executed. When an operation of selecting one piece of workout information displayed in the lower portion of the calendar is executed, the display processing unit 130 may set the corresponding workout information as a target and display the same detailed screen as that of FIG. 11 (screen transition of A6 →A3).

The embodiments and the modification examples to which the invention is applied have been described, but the invention is not limited to the embodiments or the modification examples without change. In an embodiment phase, constituent elements can be modified and embodied within the scope of the invention without departing from the gist of the invention. By appropriately combining the plurality of constituent elements disclosed in the foregoing embodiments and modification examples, the invention can be realized variously. For example, some of the constituent elements may be omitted from all of the constituent elements described in the embodiments or the modification examples. Further, the constituent elements described in the different embodiments and modification examples may be appropriately combined. In the specification or the drawings, the terms described at least once along with different terms which are broader or synonymous may be substituted with different terms in any portion of the specification or the drawings. In this way, various modifications or applications can be made within the scope of the invention without departing from the gist of the invention.

The scheme according to the embodiment can be applied to a workout information display method in which the above-described sequences are executed. Here, the sequences include the display sequences described in the specification and the drawings or the sequences in which the operations by the user are received. For example, the scheme according to the embodiment can be applied to the workout information display method in which the screen transition of FIG. 5 or the display of the screens of FIGS. 6, 8 to 16, 20, and 21 are performed.

What is claimed is:

1. A workout information display method for use by a user, the workout information display method comprising:
acquiring workout information based on a workout executed by the user;
displaying, on at least one display, a graph in which a first axis represents a distance or a time and a second axis represents a measurement value of each measurement item of the workout information in a first display region of the at least one display;
displaying, on the at least one display, first to P-th icons (where P is an integer equal to or greater than 2) for selecting setting of measurement values of first to P-th measurement items on the second axis of the graph in a second display region of the at least one display when there are the first to P-th measurement items as the measurement items; and
displaying, in the at least one display, a measurement value of the measurement item corresponding to the icon in regard to at least one of the first to P-th icons.

2. The workout information display method according to claim 1, wherein
when a point or a range is designated in the display of the graph, the first to P-th measurement values in the designated point or range are displayed in regard to the first to P-th icons, and
when no point or no range is designated in the display of the graph, statistical values obtained through a statistical process for the first to P-th measurement values during a workout period are displayed in regard to the first to P-th icons.

3. The workout information display method according to claim 1, wherein the first to P-th measurement values include at least one measurement value of a lap, a pace, a pitch, a stride, altitude information, a heart rate, and consumed calories in the workout.

4. The workout information display method according to claim 1, wherein
an operation by the user is received and the graph and an adjustment object for changing a range of the graph displayed in the first display region are displayed in the first display region side by side,
a length from a starting point to an ending point of the range is changed by changing at least one of the starting point and the ending point of the range when a first operation by the user on the adjustment object is received, and
the starting point and the ending point are changed while keeping the length from the starting point to the ending point of the range when a second operation by the user on the adjustment object is received.

5. The workout information display method according to claim 1, wherein map information associated with the workout is displayed in a third display region of the at least one display.

6. The workout information display method according to claim 5, wherein lap information of the workout is displayed in a fourth display region of the at least one display.

7. The workout information display method according to claim 6, wherein the first, second, third, and fourth display regions are arranged on only one display screen.

8. The workout information display method according to claim 6, wherein the fourth display region is disposed to overlap the third display region.

9. The workout information display method according to claim 8, wherein the fourth display region is switched between non-display and the overlap display on the third display region in response to the received operation by the user.

10. The workout information display method according to claim 6, wherein when an expansion operation by the user is received, the third display region is expanded to be displayed and the first and second display regions are moved in a predetermined direction.

11. The workout information display method according to claim 10, wherein when the third display region is expanded to be displayed, at least one of the number of pieces of lap information and an amount of information displayed in the fourth display region is increased further than when the third display is not expanded to be displayed.

12. A workout information display method for use by a user, the workout information display method comprising:
acquiring map information associated with a workout executed by the user and lap information of the workout as workout information based on the workout; and
disposing and displaying, in at least one display screen, the lap information to overlap the map information.

13. The workout information display method according to claim 12, wherein the lap information is switched between non-display and the overlap display on the map information in response to the received operation by the user.

14. A workout information display system for use by a user, the workout information display system comprising:
a reception unit that receives an operation by the user;
a storage unit that stores workout information based on a workout executed by the user; and
a display unit that displays the workout information, wherein
the display unit displays a graph in which a first axis represents a distance or a time and a second axis represents a measurement value of each measurement item of the workout information in a first display region,
the display unit displays first to P-th icons (where P is an integer equal to or greater than 2) for selecting setting of measurement values of first to P-th measurement items on the second axis of the graph in a second display region when there are the first to P-th measurement items as the measurement items, and
the display unit displays a measurement value of the measurement item corresponding to the icon in regard to at least one of the first to P-th icons.

15. A workout information display system for use by a user, the workout information display system comprising:
a reception unit that receives an operation by the user;
a storage unit that stores workout information based on a workout executed by the user; and
a display unit that displays the workout information, wherein
the display unit disposes lap information of the workout to overlap map information associated with the workout.

16. A server system for use by a user, the server system comprising:
a reception unit that receives operation information by the user;
a storage unit that stores workout information based on a workout executed by the user; and
a display processing unit that performs a process of displaying the workout information on a display unit, wherein
the display processing unit performs a process of displaying a graph in which a first axis represents a distance or a time and a second axis represents a measurement value of each measurement item of the workout information in a first display region,
the display processing unit performs a process of displaying first to P-th icons (where P is an integer equal to or greater than 2) for selecting setting of measurement values of first to P-th measurement items on the second axis of the graph in a second display region when there are the first to P-th measurement items as the measurement items, and
the display processing unit performs a process of displaying a measurement value of the measurement item corresponding to the icon in regard to at least one of the first to P-th icons.

17. A server system for use by a user, the server system comprising:
a reception unit that receives an operation by the user;
a storage unit that stores workout information based on a workout executed by the user; and
a display processing unit that performs a process of displaying the workout information on a display unit, wherein
the display processing unit performs a process of displaying lap information of the workout to overlap map information associated with the workout.

18. An electronic device comprising:
the workout information display system according to claim 14.

19. An information storage medium storing a program for use by a user, the program causing a computer to function as:
a reception unit that receives an operation by the user;
a storage unit that stores workout information based on a workout executed by the user; and
a display processing unit that performs a process of displaying the workout information on a display unit, wherein
the display processing unit displays a graph in which a first axis represents a distance or a time and a second axis represents a measurement value of each measurement item of the workout information in a first display region,
the display processing unit displays first to P-th icons (where P is an integer equal to or greater than 2) for selecting setting of measurement values of first to P-th measurement items on the second axis of the graph in a second display region when there are the first to P-th measurement items as the measurement items, and
the display processing unit displays a measurement value of the measurement item corresponding to the icon in regard to at least one of the first to P-th icons.

20. An information storage medium storing a program for use by a user, the program causing a computer to function as:
a reception unit that receives an operation by the user;
a storage unit that stores workout information based on a workout executed by the user; and
a display processing unit that performs a process of displaying the workout information on a display unit, wherein
the display processing unit disposes lap information of the workout to overlap map information associated with the workout.

21. A workout information display system for use by a user, the workout information display system comprising:
a transmitter/receiver receiving an operation by the user;
a memory storing workout information based on a workout executed by the user;
a display displaying the workout information; and
a processor programmed to:
display, on the display, a graph in which a first axis represents a distance or a time and a second axis represents a measurement value of each measurement item of the workout information in a first display region,
display, on the display, first to P-th icons (where P is an integer equal to or greater than 2) for selecting setting of measurement values of first to P-th measurement items on the second axis of the graph in a second display region when there are the first to P-th measurement items as the measurement items, and
display, on the display, a measurement value of the measurement item corresponding to the icon in regard to at least one of the first to P-th icons.

22. A workout information display system for use by a user, the workout information display system comprising:
a transmitter/receiver receiving an operation by the user;
a memory storing workout information based on a workout executed by the user;
a display displaying the workout information; and
a processor programmed to:
display lap information of the workout to overlap map information associated with the workout.

23. A electronic device comprising:
the workout information display system according to claim 21.

24. A electronic device comprising:
the workout information display system according to claim 22.

25. A server system for use with a display and by a user, the server system comprising:
a transmitter/receiver receiving operation information by the user;
a memory storing workout information based on a workout executed by the user; and
a processor programmed to:
display, on the display, a graph in which a first axis represents a distance or a time and a second axis represents a measurement value of each measurement item of the workout information in a first display region,
display, on the display, first to P-th icons (where P is an integer equal to or greater than 2) for selecting setting of measurement values of first to P-th measurement items on the second axis of the graph in a second display region when there are the first to P-th measurement items as the measurement items, and
display, on the display, a measurement value of the measurement item corresponding to the icon in regard to at least one of the first to P-th icons.

26. A server system for use with a display and by a user, the server system comprising:
a transmitter/receiver receiving an operation by the user;
a memory storing workout information based on a workout executed by the user; and
a processor programmed to:
display, on the display, lap information of the workout to overlap map information associated with the workout.
